(19) United States
(12) Patent Application Publication
(10) Pub. No.: US 2022/0010269 A1
(43) Pub. Date: Jan. 13, 2022

(54) Engineering Of Innervated Tissue And Modulation Of Peripheral Organ Activity

(71) Applicants: THE TRUSTEES OF THE UNIVERSITY OF PENNSYLVANIA, Philadelphia, PA (US); The United States of America as Represented by the Department of Veterans Affairs, Washington, DC (US)

(72) Inventors: Daniel Kacy Cullen, Media, PA (US); Suradip Das, Philadelphia, PA (US); Wisberty Gordian-Velez, Philadelphia, PA (US)

(21) Appl. No.: 17/291,869

(22) PCT Filed: Nov. 8, 2019
(86) PCT No.: PCT/US2019/060585

§ 371 Date: May 6, 2021

Related U.S. Application Data
(60) Provisional application No. 62/758,203, filed on Nov. 9, 2018.

(51) Int. Cl. C12N 5/00; C12N 5/077; C12N 5/071; C12N 5/0793; C12N 5/078; A61K 35/34; A61P 21/00

(52) U.S. Cl. CPC C12N 5/0068; C12N 2533/80; C12N 5/0677; C12N 5/0679; C12N 5/0633; C12N 5/0619; C12N 5/0658; C12N 5/0648; C12N 5/0685; A61K 35/34; A61P 21/00; C12N 2502/081; C12N 2513/00; C12N 2533/76; C12N 5/0657

(57) ABSTRACT

In various aspects and embodiments, the present invention provides methods for preparing innervated tissue. In various embodiments the invention further provides innervated tissue generated using the methods described herein. In various embodiments the inclusion of optogenetically transducible TENGs or Micro-TENNs in the innervated tissue allows the modulation of tissue or organs by using light to stimulate the optogenetically transducible TENGs or Micro-TENNs.

### CROSS REFERENCE TO RELATED APPLICATIONS

The present application claims priority under 35 U.S.C. § 119(e) to U.S. Provisional Patent Application No. 62/758,203 filed Nov. 9, 2018, which is incorporated herein by reference in its entirety.

### STATEMENT REGARDING FEDERALLY SPONSORED RESEARCH OR DEVELOPMENT

This invention was made with government support under grant number 101-BX003748 awarded by the Department of Veterans Affairs and grant number W81XWH-16-1-0796 awarded by the Department of Defense. The government has certain rights in the invention.

### BACKGROUND OF THE INVENTION

The nervous system is intimately connected to all tissue and organs by virtue of the physical coupling of axonal terminals with specialized cells in the end organ. As a result, innervation plays a pivotal role as both a driver of tissue/organ development and as a means for their functional control and modulation. Indeed, innervation-induced developmental mechanisms are vital to direct the proper form and function of tissues/organs, and therefore the crucial role of innervation should be considered throughout the entire process of fabricating engineered tissues and organs. In addition, while many organs function independently from direct voluntary inputs, they are generally under precise autonomic regulation. Unfortunately, the role of innervation has generally been overlooked in most non-neural tissue engineering applications. To innervate engineered tissues and organs, it is not simply a matter of hooking up two opposing ends; rather, specific host axon populations often need to be precisely driven to appropriate location(s) within the construct, often over long distances. There is a need in the art for improvements in tissue engineering. This disclosure addresses that need.

### SUMMARY OF THE INVENTION

In one aspect, the invention provides a method of generating innervated cardiac tissue, the method comprising:

a) isolating cardiac myocytes;

b) culturing the cardiac myocytes on a first scaffold;

c) isolating and culturing sympathetic ganglia and parasympathetic neurons from cervical ganglia and intracardiac ganglia;

d) co-culturing parasympathetic neurons with the cardiac myocytes on the first scaffold;

e) culturing the sympathetic ganglia on a second scaffold adjacent to the first scaffold, thereby forming a construct;

f) maturing the construct in a bioreactor; thereby generating innervated cardiac tissue.

In another aspect, the invention provides a method of generating innervated tissue engineered pancreatic tissue, the method comprising:

a) isolating pancreatic acinar and beta islet cells;

b) culturing the pancreatic acinar cells and beta islet cells on a first scaffold;

c) isolating and culturing sympathetic ganglia and parasympathetic neurons;

d) co-culturing parasympathetic neurons with the pancreatic acinar cells and beta islet cells on the first scaffold;

e) culturing the sympathetic ganglia on a second scaffold adjacent to the first scaffold, thereby forming a construct;

f) maturing the construct in a bioreactor;
thereby generating innervated pancreatic tissue.

In another aspect, the invention provides a method of generating innervated intestinal tissue, the method comprising:

a) isolating intestinal smooth muscle cells;

b) culturing the intestinal smooth muscle cells on a first scaffold;

c) isolating and culturing enteric neurons;

d) co-culturing enteric neurons with the intestinal smooth muscle cells on the first scaffold, thereby forming a construct;

e) maturing the construct in a bioreactor; thereby generating innervated intestinal tissue.

In another aspect, the invention provides a method of generating innervated salivary gland tissue, the method comprising:

a) isolating salivary acinar cells;

b) culturing the salivary acinar cells on a first scaffold;

c) isolating and culturing sympathetic and parasympathetic neurons;

d) culturing sympathetic neurons on a second scaffold, culturing parasympathetic neurons on a third scaffold, wherein the second scaffold and the third scaffold are adjacent to the first scaffold, thereby forming a construct;

e) maturing the construct in a bioreactor; thereby generating innervated salivary gland tissue.

In another aspect, the invention provides a method of generating innervated skeletal muscle tissue, the method comprising:

a) isolating skeletal myocytes;

b) culturing the skeletal myocytes on a first scaffold to form myofibers;

c) isolating spinal motor neurons;

d) co-culturing the motor neurons with the myofibers on the first scaffold, thereby forming a construct;

e) maturing the construct in a bioreactor; thereby generating innervated skeletal muscle tissue.

In another aspect, the invention provides a method of generating innervated spleen tissue, the method comprising:

a) isolating sympathetic neurons;

b) culturing the sympathetic neurons on a first scaffold while allowing axonal growth to an adjacent second scaffold;

c) isolating splenocytes;

d) co-culturing the splenocytes on the first scaffold with the sympathetic neurons;

e) maturing the construct in a bioreactor; thereby generating innervated spleen tissue.

In another aspect, the invention provides a method of generating innervated bladder tissue, the method comprising:

a) isolating bladder smooth muscle cells and urothelial cells;

b) co-culturing the bladder smooth muscle cells and the urothelial cells on a first scaffold;

c) isolating sympathetic neurons and parasympathetic neurons;

d) culturing the sympathetic neurons on a second scaffold and the parasympathetic neurons on a third scaffold, wherein the second and third scaffolds are adjacent to the first scaffold, thereby forming a construct;

e) maturing the construct in a bioreactor; thereby generating innervated bladder tissue.

In various embodiments, at least one scaffold comprises a living scaffold.

In various embodiments, the invention provides innervated tissue generated according to the methods described herein.

In various embodiments, the innervated tissue, comprises at least one TENG or Micro-TENN.

In various embodiments, the invention provides a method of treating a disease or disorder in a subject, the method comprising implanting innervated tissue made according to the methods of the invention into the subject.

In various embodiments, the invention provides a method of treating a disease or disorder in a subject, the method comprising implanting the tissue of the invention into the subject and wiring the at least one TENG or Micro-TENN to at least one native neuron of the subject.

In various embodiments, the at least one TENG or Micro-TENN is an optogenetically-transducible TENG or Micro-TENN.

In various embodiments, the invention provides a method of modulating a tissue or organ of a subject, the method comprising implanting innervated tissue of the invention, into the subject and applying light to activate the optogenically transducible TENG or micro-TENN.

In another aspect, the invention provides a method of generating innervated cardiac tissue, the method comprising:

a) providing a micro-column having a first end and a second end, and comprising a tubular hydrogel body and an extracellular matrix core;

b) positioning cardiac myocyte aggregates at the first end of the micro-column and positioning sympathetic neuron aggregates at the second end of the micro-column, thereby forming a construct;

c) culturing the construct in vitro to promote extension of an axon of the neuron as well as the cardiac myocytes through at least a portion of the core, thereby generating innervated cardiac tissue.

In various embodiments, the tubular body comprises at least one selected from the group consisting of hyaluronic acid, chitosan, alginate, collagen, dextran, pectin, carrageenan, polylysine, gelatin and agarose.

In various embodiments, the tubular body comprises methacrylated hyaluronic acid.

In various embodiments, the extracellular matrix core comprises collagen, fibronectin, fibrin, hyaluronic acid, elastin, and laminin.

In various embodiments, the micro-column has a length of about 3-10 mm.

In various embodiments, the micro-column has an outer diameter from about 500 μm to about 1 mm.

In various embodiments, the micro-column has an inner diameter from about 125 μm to about 500 μm.

In another aspect, the invention provides a method of generating innervated skeletal muscle tissue, the method comprising:

a) culturing skeletal myocytes on a substrate comprising nanofibers aligned in a first direction, thereby forming a myocyte layer;

b) co-culturing motor neurons on the myocyte layer; thereby generating innervated skeletal muscle tissue.

In various embodiments, the substrate comprises polycaprolactone.

In various embodiments, the method further comprises

c) applying a tensile force perpendicular to the first direction.

In various embodiments, the tensile force is applied at a rate of about 0.1 mm/day.

In various embodiments, the tensile force is applied for about 5 days to achieve a net stretch of about 0.5 mm.

In various embodiments, the cardiac myocytes are mammalian cardiac myocytes.

In various embodiments, the cardiac myocytes are human cardiac myocytes.

In various embodiments, the skeletal myocytes are mammalian skeletal myocytes.

In various embodiments, the skeletal myocytes are human skeletal myocytes.

In various embodiments, the invention provides a method of treating a muscle injury in a subject in need thereof, the method comprising contacting the muscle injury with innervated skeletal muscle tissue generated by the methods of the invention.

In various embodiments, the invention provides a method of modeling development, maturation, function, injury, and/or disease, the method comprising using the innervated engineered tissue generated according to the methods of the invention as an in vitro testbed.

### BRIEF DESCRIPTION OF THE DRAWINGS

The following detailed description of preferred embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1 depicts an overview of methods for the fabrication of innervated bioengineered organs/tissues.

FIG. 2 illustrates an embodiment for generating tissue engineered myocardium. Step 1 Isolation of cardiac myocytes from murine or human sources; Step 2 Culture and maintenance of cardiac myocytes on an appropriate 3 dimensional (3D) scaffold; Step 3 Isolation and primary culture of sympathetic and parasympathetic populations from superior cervical ganglia and intracardiac ganglia respectively; Step 4 Co-culture of parasympathetic neurons with cardiac myocytes growing on scaffold and Step 5 Culture of sympathetic ganglia on a separate adjacent scaffold to mimic native architecture; Step 6 Maturation of whole construct in an appropriate bioreactor.

FIG. 3 illustrates an embodiment for generating innervated tissue engineered pancreas. Step 1 Isolation and primary culture of pancreatic acinar and beta islet cells from murine or human sources; Step 2 Co-culture of acinar and islet cells on an appropriate 3D scaffold; Step 3 Isolation and primary culture of sympathetic and parasympathetic populations from celiac ganglia and submandibular ganglia respectively; Step 4 Plating and culture of sympathetic and parasympathetic ganglia on separate scaffolds adjacent to the scaffold containing pancreatic cell population; Step 5 Upon adequate axonal infiltration into the pancreatic cells from adjacent autonomic ganglia the whole setup may be transferred for culture and maturation in a bioreactor.

FIG. 4 illustrates an embodiment for generating innervated tissue engineered intestine. Step 1 Isolation and primary culture of intestinal smooth muscle cells from murine or human sources; Step 2 Culture of intestinal smooth muscle cells on an appropriate 3D scaffold; Step 3 Isolation and primary culture of enteric neurons from the myenteric plexus; Step 4 Plating and culture of enteric neurons on scaffold containing smooth muscle cells; Step 5 Following innervation of the smooth muscle cells with enteric neurons, the construct may be allowed to mature in a bioreactor.

FIG. 5 illustrates an embodiment for generating innervated salivary gland tissue. Step 1 Isolation and primary culture of salivary acinar cells from any of the 3 salivary glands depending upon the target organ; Step 2 Culture of acinar cells on an appropriate 3D scaffold; Step 3 Isolation and primary culture of sympathetic and parasympathetic populations from superior cervical ganglia and submandibular ganglia respectively; Step 4 Plating and culture of sympathetic and parasympathetic ganglia on separate scaffolds adjacent to the scaffold containing salivary acinar cell population; Step 5 Upon adequate axonal infiltration into the salivary acinar cells from adjacent autonomic ganglia the whole setup may be transferred for culture and maturation in a bioreactor.

FIG. 6 illustrates an embodiment for generating innervated tissue engineered skeletal muscle. Step 1 Isolation and primary culture of skeletal myocytes from murine or human sources; Step 2 Culture of skeletal myocytes on an appropriate 3D scaffold to form mature myofibers; Step 3 Isolation of spinal motor neurons followed by Step 4 forced aggregation and formation of 3D motor neuron aggregates. Step 5 The motor neuron aggregates may be plated on a bed of pre-differentiated myofibers grown on a scaffold and co-cultured. Step 6 Following formation of adequate neuromuscular connections the construct may be allowed to mature in a bioreactor.

FIG. 7 illustrates an embodiment for generating innervated tissue engineered spleen. Step 1 Isolation and primary culture of sympathetic neurons from the celiac ganglia; Step 2 Plating and culture of sympathetic neurons on a scaffold and allowing axonal outgrowth into an adjacent scaffold Step 3 Isolation and primary culture of splenocytes; Step 4 Culture of splenocytes on the scaffold which already consists of sympathetic neurons; Step 5 Following innervation of the splenocytes with sympathetic neurons, the construct may be allowed to mature in a bioreactor.

FIG. 8 illustrates an embodiment for generating innervated tissue engineered bladder. Step 1 Isolation and primary culture of bladder smooth muscle and urothelial cells from separate layers of the bladder wall; Step 2 Culture of bladder smooth muscle cells on an appropriate 3D scaffold (A) followed by seeding and culture of urothelial cells (B); Step 3 Isolation and primary culture of parasympathetic populations from submandibular ganglia. The sympathetic neurons may be harvested from either pelvic or celiac ganglia; Step 4 Plating and culture of sympathetic and parasympathetic ganglia on separate scaffolds adjacent to the scaffold containing the bladder cell population; Step 5 Upon adequate axonal infiltration into the bladder cells from adjacent autonomic ganglia the whole setup may be transferred for culture and maturation in a bioreactor.

FIGS. 9A and 9B depict the application of micro-TENNs for muscle reinnervation and as neuromodulatory interfaces. FIG. 9A shows that Micro-TENNs may serve to promote regeneration of axonal connections from spinal motor neurons to muscles suffering volumetric muscle loss (VML). In this application, micro-TENNs can be precisely microinjected at the proximal nerve and with engineered muscle distally to guide axon growth from the nerve back to the muscle belly to regenerate lost neuromuscular connections. FIG. 9B shows that artificial autonomic ganglia can be comprised of light-activated sympathetic or parasympathetic neuron aggregates extending axon tracts within a hydrogel encasement and may be constructed as parallel pathways to native autonomic innervation. These living constructs would project axons to innervate the target organ, thereby providing a light and computer-controlled, yet natural, source of norepinephrine (NE) or acetycholine (ACh) for selective modulation of organ function.

FIGS. 10A-10D: Characterization of growth and phenotype of engineered axonal tracts projected from sympathetic aggregates. FIG. 10A: Pieces of SCG isolated from postnatal rats were cultured as aggregates in laminin-coated 2D surfaces for 10 DIV and stained for neurons/axons (Tuj1/β-tubullin III), noradrenergic neurons (TH), and nuclei (Hoechst). The staining confirmed that the neurons and axons exhibit the correct TH+ phenotype expected from sympathetic neurons. FIGS. 10B and 10C: The neurite growth length and rate were quantified for neurites extended by sympathetic aggregates cultured within 3D MeHA hydrogel columns as a function of time. Repeated measures ANOVA yielded a significant effect of time on neurite growth and rate with p<0.0001 and p=0.0381, respectively. Data presented as mean±SEM with individual values included (*p<0.05, **p<0.01, ***p<0.001, ****p<0.0001). FIG. 10D: Phase-contrast images of a representative 3D MeHA hydrogel column containing a sympathetic aggregate showing the progression of neurite growth with DIV. Scale bars: 500 μm.

FIGS. 11A-11E: Co-culture of cardiac myocyte aggregates with SCG-derived sympathetic aggregates within a 3D hydrogel micro-column augments beating rate. FIG. 11A: Phase-contrast image of a ˜3 mm MeHA hydrogel micro-column containing sympathetic and cardiac aggregates on the left and right end, respectively, at 6 DIV. The two populations are connected by dense axonal tracts emanating from the sympathetic aggregate. FIG. 11B: Confocal reconstruction of the cardiac aggregate region of the construct in A stained to denote cardiac myocytes (troponin), neurons/axons (Tuj1/β-tubullin III), noradrenergic neurons (TH), and nuclei (Hoechst). The close physical proximity between Tuj1+/TH+ neurites and troponin+ cardiac myocytes suggests that as the sympathetic aggregates extended neurites, they reached the cardiac aggregate to innervate it. FIG. 11C: Hydrogel micro-columns were also fabricated with only a cardiac myocyte population on one end as a control, as shown by this phase-contrast image of a construct at 5 DIV. FIG. 11D: The cardiac-only construct was stained for the same markers as B, showing the cytoarchitecture of the cardiac aggregate. FIG. 11E: The contraction rate of the cardiac aggregates was analyzed as the number of beats per minute at 5 and 8 DIV with micro-columns containing both sympathetic and cardiac aggregates (co-culture; circle markers) and only cardiac myocytes (square markers). The results suggest that the presence of sympathetic aggregates significantly augments the spontaneous beating rate of 3D cardiac aggregates relative to cardiac-only controls at both 5 and 8 DIV. Moreover, the ANOVA test produced a significant effect of culture type on contraction rate with p=0.0038. Data presented as mean±SEM with individual values included (*p<0.05). Scale bars: (FIGS. 11A, 11C) 400 μm, (FIG. 11B) 100 μm, (FIG. 11D) 500 μm.

FIGS. 12A-12E: Development of Mechanically Stretch Grown Innervated Tissue Engineered Muscle Construct FIGS. 12A-12C: Custom build mechanical bioreactors control tensile forces to simulate functional muscle mechanisms in physiological conditions. FIG. 12D: Before and after a 0.5 cm stretch operation. FIG. 12E: Myocytes are differentiated on aligned nanofiber scaffolds to form thick myotubes, responding differently to the alignment of the nanofibers with respect to the direction of stretch. Spinal motor neurons and skeletal myocytes are co-cultured on the same sheet by which the neuromuscular bundle orients perpendicular to the direction of stretch. The towing membrane is gradually pulled by the stepper motor to instigate “stretch growth”. Skeletal myocytes were plated at the density of 400,000 myocytes per sheet and spinal motor neurons were 100,000 motor neurons plated per sheet. Stretching perpendicular to the nanofiber alignment enhanced fusion of myofibers as compared to when they were stretched parallel to the alignment of the nanofibers.

FIG. 13: Mechanical properties of nanofiber scaffolds according to direction of stretch. Electrospun nanofiber polycaprolactone (PCL) sheets soaked in distilled water for 1 or 2 weeks to measure degradation in tensile strength. Tensile testing was performed using the Instron Model 5544 at 0.02 mm/sec at either time point and the curves indicate the loss of tensile properties of parallel, and perpendicularly aligned nanofibers with respect to the direction of stretch.

FIGS. 14A-14C: Effect of Mechanical Stretch Direction on Neuromuscular Alignment. Motor neurons and myocytes were observed to orient perpendicular to direction of mechanical force. FIG. 14A: F-actin in skeletal myotubes and β-tubulin III in motor axons are stained with phalloidin and Tuj-1 respectively. Myocytes and motor axons appear dispersed and oriented at an angle to the fiber direction when stretched parallel to the nanofiber alignment. FIGS. 14B-14C: Skeletal myocytes and motor neurons formed aligned dense bundles along the nanofiber alignment when stretched perpendicularly to direction of stretch.

FIGS. 15A-15B: Concept of Pre-Innervated Tissue Engineered Muscle. The present study was focused on exploring the role of pre-innervation on myocytes in vitro and host neuromuscular environment in vivo following implantation. FIG. 15A: For in vitro studies, our overarching hypothesis were that innervation would augment skeletal myocyte fusion, maturation and formation of Neuromuscular Junctions (NMJs). FIG. 15B: Volumetric Muscle Loss (VML) is defined as frank loss of muscle volume that is accompanied by chronic motor axotomy leading to denervation of the injured area. We used a standardized rat model of VML where >20% of the Tibialis Anterior (TA) muscle volume was resected to create a defect leading to potential damage to intramuscular branches of the host nerve and loss of motor end plates (or NMJs) near the injury area. For in vivo studies, our overarching hypothesis were that implantation of pre-innervated constructs would enhance Acetylcholine Receptor (AchR) clustering and promote innervation of AchRs (mature NMJs) near the implant site at acute time point.

FIGS. 16A-16E: Co-culture of Motor Neuron-Myocyte on Aligned Nanofiber Scaffolds. FIG. 16A: Skeletal myoblast cell line C2C12 was cultured and allowed to differentiate on aligned nanofiber scaffolds. Mature myofibers were observed to align along the nanofibers when stained for F-Actin (Phalloidin-488). Scale bar—200 μm. FIG. 16B: Spinal motor neurons cultured on the nanofiber scaffolds aligned along the direction of nanofibers as observed by staining with pan-axonal marker Tuj-1. Scale bar—200 μm. FIG. 16C and FIG. 16D: Co-culture of motor neurons and myocytes on the nanofiber scaffolds resulted in formation of aligned neuromuscular bundles as observed by labelling for F-Actin (Phalloidin-488) (FIG. 16D) and Tuj-1 (FIG. 16E). Scale bar—100 μm.

FIGS. 17A-17C: Innervation of Myocytes and Effect of Motor Neurons on Myocyte Maturation In Vitro. FIG. 17A: Rat spinal motor neurons were introduced on a bed of myofibers differentiated on aligned nanofiber sheet for 7 days and subsequently co-cultured for another 7 days leading to innervation of the skeletal myofibers. Scale bar—100 μm. The area marked a′ is a higher magnification view of the area marked by white box reveals structures colabelling for presynaptic marker (Synaptophysin) and Acetylcholine Receptor (AchR) clusters (Bungarotoxin) indicating formation of mature neuromuscular junctions in vitro (indicated by white arrows). Scale bar—50 μm. FIG. 17B: Myocytes exhibited greater fusion and bundling when co-cultured with motor neurons (MN-MYO) as compared to monoculture (MYO). Scale bar—100 μm. FIG. 17C: Myocyte Fusion Index (MFI) was calculated from multiple cultures (n≥6), and co-culture with motor neurons was found to significantly enhance MFI. For indicated comparison: p≤0.0001 (****). Error bars represent standard error of mean.

FIGS. 18A-18F: Bio-Scaffold Implantation in VML Model. FIGS. 18A-18B: Surgical resection of TA muscle to create VML model in rats. FIG. 18C: Implant of cell-laden nanofiber sheets in muscle defect. Scaffold and overlying fascia secured with sutures. FIGS. 18D-18F: At 7 days post-implant, animals were sacrificed, and TA muscle was excised. Nanofiber sheets were seen in Repair Group (FIG. 18E) whereas injury site was recessed in No Repair group (FIG. 18F).

FIGS. 19A-19D: Acute Survival of Implanted Cells In Vivo. FIGS. 19A-19D: Cross section images of the repair/injury site from animals implanted with nanofibers comprised of FIG. 19A: motor neurons+myocytes (MN-MYO), FIG. 19B: myocytes only (MYO), FIG. 19C: acellular Sheets and FIG. 19D: No Repair. The sections showed presence of axons (ChAT+ & NF-200+) on the nanofiber sheets in the MN-MYO group whereas the other groups were negative for axonal markers at the injury site. The nanofiber sheets on both the MN-MYO and MYO groups showed presence of Phalloidin+myocytes. Dashed lines indicate margins of nanofiber sheets. Scale bar—200 μm.

FIGS. 20A-20D: Cellular and Morphological Evaluation of Pre-Innervated Constructs at Acute Time Point Following Implantation in a VML Model. FIGS. 20A-20D: Longitudinal sections near the repair site of animals implanted with nanofibers with motor neurons+myocytes (MN-MYO). The nanofiber sheets were coated with Laminin prior to culturing cells and hence the stacked sheets were identified based on Laminin stain (FIG. 20C). Scale: 500 μm. a′-d′) Magnified view of the region inside the white box. Thick bundles of myocytes (Phalloidin: FIG. 20B) and motor axons (NF-200: FIG. 20D) were observed within the stacked nanofiber sheets. FIG. 20A: all markers. Scale: 100 μm.

FIGS. 21A-21F: Satellite cell migration near injury area following VML. FIGS. 21A-D: Muscle satellite cells near the injury area were identified by staining for satellite cell marker—Pax 7 in FIG. 21A: MN-MYO; FIG. 21B: MYO; FIG. 21C: Sheets; FIG. 21D: No repair groups. Scale bar—100 μm. FIG. 21E: Representative image of a higher magnification view of satellite cells. Pax 7+ nuclei located on the periphery of Skeletal Muscle Actin+ myofiber and colabelling with pan-nuclear marker DAPI were identified as satellite cells. Scale bar—10 μm. FIG. 21F: Satellite cell density near the injury area (5 mm2) was counted across MN-MYO (n=5), MYO (n=4), Sheets (n=3) and No Repair (n=5) groups. Mean satellite cell density of each group were as follows: MN-MYO—141.4; MYO—64.06; Sheets—67.75; No Repair—66.85. For indicated comparisons the individual p-values were as follows: MN-MYO vs MYO—p=0.0029 (**); MN-MYO vs Sheets—p=0.0081 (**); MN-MYO vs No Repair—p=0.0024 (**). Error bars represent standard error of mean.

FIGS. 22A-22F: Micro-vessel density near injury area following VML. FIGS. 22A-22D: Endothelial cells and micro-vasculature near the injury area were identified by staining for endothelial cell marker—CD31 and Smooth Muscle Actin in MN-MYO (FIG. 22A); MYO (FIG. 22B); Sheets (FIG. 22C); No repair groups (FIG. 22D). Scale bar—200 μm. FIG. 22E: Representative image of a higher magnification view of endothelial cells and micro-vessels. Structures expressing CD31 and Smooth Muscle Actin with a visible lumen and an area >50 μm² were defined as micro-vessels. Scale bar—10 μm. FIG. 22F: Micro-vessel density near the injury area (5 mm2) was counted across MN-MYO (n=5), MYO (n=4), Sheets (n=3) and No Repair (n=5) groups. Mean microvessel density of each group were as follows: MN-MYO—40.84; MYO—17.7; Sheets—18.47; No Repair—25.76. For indicated comparisons the individual p-values were as follows: MN-MYO vs MYO—p=0.0024 (**); MN-MYO vs Sheets—p=0.0061 (**); MN-MYO vs No Repair—p=0.0321 (*). Error bars represent standard error of mean.

FIGS. 23A-23F: Acetylcholine Receptor (AchR) Clusters near injury area following VML. FIGS. 23A-23D: AchR clusters near the injury area were identified by staining with Bungarotoxin in FIG. 23A: MN-MYO; FIG. 23B: MYO; FIG. 23C: Sheets; FIG. 23D: No repair groups. Scale bar—500 μm. FIG. 23E: Representative image of a higher magnification view of pretzel shaped AchR clusters on the periphery of muscle fibers (Phalloidin-488). Scale bar—50 μm. FIG. 23F: AchR cluster density near the injury area (5 mm2) was counted across MN-MYO (n=5), MYO (n=4), Sheets (n=3) and No Repair (n=5) groups. Mean AchR cluster density of each group were as follows: MN-MYO—5.92; MYO—2.167; Sheets—2.311; No Repair—3.227. For indicated comparisons the individual p-values were as follows: MN-MYO vs MYO—p<0.0001 (****); MN-MYO vs Sheets—p=0.0001 (***); MN-MYO vs No Repair—p=0.0006 (***). Error bars represent standard error of mean.

FIGS. 24A-24F: Pre-Innervation promotes mature NMJs formation near injury area following VML. FIGS. 24A-24D: Mature NMJs near the injury area were identified by double staining with Bungarotoxin and presynaptic marker Synaptophysin in FIG. 24A: MN-MYO; FIG. 24B: MYO; FIG. 24C: Sheets; FIG. 24D: No repair groups and are indicated by yellow stars. Scale bar—100 μm. FIG. 24E: Representative image of a higher magnification view of mature NMJs (indicated by stars) comprising of pretzel shaped AchR clusters colabelling with presynaptic marker Synaptophysin located on the periphery of muscle fibers (Phalloidin-488). Scale bar—10 μm. FIG. 24F: Percentage of AchR clusters near the injury area (5 mm2) that were innervated (Synaptophysin+) was counted across MN-MYO (n=5), MYO (n=4), Sheets (n=3) and No Repair (n=5) groups to depict maintenance/formation of mature NMJs in the host muscle. Mean percentage of mature NMJ of each group were as follows: MN-MYO—78.95; MYO—52.9; Sheets—38.77; No Repair—20.2. For indicated comparisons the individual p-values were as follows: MN-MYO vs MYO—p=0.0168 (*); MN-MYO vs Sheets—p=0.0012 (**); MN-MYO vs No Repair—p<0.0001 (****). Error bars represent standard error of mean.

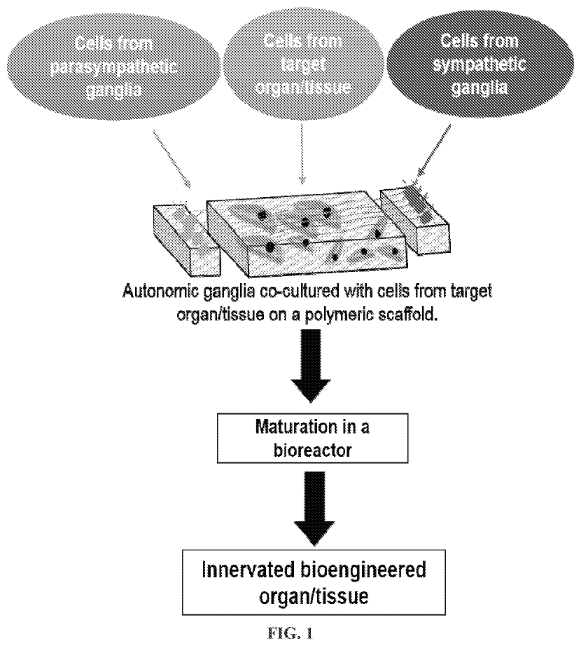

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting. As used herein, the singular form “a,” “an,” and “the” include plural references unless the context clearly dictates otherwise.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

“Isolating,” means to obtain one or more types of cells, purify to remove or substantially remove other cells types and grow in primary culture.

A “subject” or “patient,” as used therein, may be a human or non-human mammal. Non-human mammals include, for example, livestock and pets, such as ovine, bovine, porcine, canine, feline and murine mammals. Preferably, the subject is human.

“Scaffold” refers to a framework upon which cells are cultured.

“Tissue engineered axonal tracts” refer to living axonal tracts generated from TENGs, in which the neuronal cell bodies have been severed leaving only axonal tracts. In various embodiments the TENG may have been generated from any sub-type of neuron, including but not limited to neurons from the peripheral nervous system (e.g., spinal motor, sensory dorsal root ganglia), central nervous system (e.g., glutamatergic, GABAergic, dopaminergic, serotonergic), and autonomic nervous system (e.g, ganglionic norepinephrinergic, acetycholinergic, or dopaminergic).

“Tissue-Engineered Nerve Grafts (TENGs)” is used interchangeably herein with the term “stretch-grown TENG” and refers to living three-dimensional nerve constructs that consist of neurons, including neuronal cell bodies, and longitudinally aligned axonal tracts.

“Forced aggregation TENG” and “forced cell aggregation TENG” are used interchangeably to refer to a TENG that is stretch grown from an aggregate or sphere of neurons formed by forced aggregation.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

In various aspects and embodiments, the present invention provides methods for preparing innervated tissue. In various embodiments the invention further provides innervated tissue generated using the methods described herein. In various embodiments the inclusion of optogenetically transducible TENGs or Micro-TENNs in the innervated tissue allows the modulation of tissue or organs by using light to stimulate the optogenetically transducible TENGs or Micro-TENNs. In various aspects and embodiments of the below described methods, the neuron source may be primary (taken from an animal) or stem cell derived. In various embodiments, neurons may be xenogeneic or allogeneic. By way of non-limiting example, rat neurons may be used in the bioreactor with human cardiomyocytes. In various embodiments, the invention further provides a method of modeling development, maturation, function, injury, and/or disease, the method comprising using the innervated engineered tissue generated according to the methods described below as an in vitro testbed. In various embodiments, the neurons may be forced aggregation neurons. i.e. neuron aggregates.

### Tissue Engineered Innervated Cardiac Tissue

In one aspect the invention provides a method of generating innervated cardiac tissue by isolating cardiac myocytes; culturing the cardiac myocytes on a first scaffold; isolating and culturing sympathetic ganglia and parasympathetic neurons from cervical ganglia and intracardiac ganglia; co-culturing parasympathetic neurons with the cardiac myocytes on the first scaffold; culturing the sympathetic ganglia on a second scaffold adjacent to the first scaffold; and maturing the construct in a bioreactor; thereby generating innervated cardiac tissue.

Parasympathetic preganglionic fibers arise from cranial nerve X (vagus nerve) in the medulla oblongata and connect in cardiac ganglia, located at the dorsal atrial surface of the heart, with postganglionic fibers that ultimately innervate the organ. These fibers notably innervate the sinoatrial (SA) and atrioventricular (AV) nodes, two crucial components of the cardiac conduction system involved in heart contraction. Sympathetic innervation of the heart consists of postganglionic fibers that enter the heart through the cardiac plexus, from cervical and thoracic ganglia in the two parallel sympathetic chains, where they synapse with preganglionic fibers from the upper thoracic spinal cord. These sympathetic fibers innervate both the circulatory and conduction systems of the heart, including smooth muscle cells (SMCs) and the SA and AV nodes, respectively.

Orthotopic heart transplantation involves implanting a donor heart that is completely disconnected from the host nervous system into a recipient. This leads to inefficient cardiac output, arrhythmia, and limited exercise tolerance. Sympathetic reinnervation following heart transplantation has been reported in a few cases after at least 3 years. Such patients were observed to have higher peak heart rate and better endurance during exercise, indicating that reinnervation aids in recovering functional capacity by improving chronotropism and inotropism. Furthermore, functional reinnervation of the transplanted heart enables angina to occur during MI which can be lifesaving. Cardiac tissue engineering aims at replacing or repairing damaged cardiac tissue using biological or polymer-based scaffolds in combination with cells and growth factors. Although the importance of electrochemical cues in engineered cardiac tissue and the role of innervation in adequate functioning of cardiac implants has been explored, the concept of fabricating an artificial cardiac tissue or whole heart with appropriate innervation has been largely unexplored. An optimal scaffold for cardiac tissue engineering should either be pre-innervated prior to implant or promote neo-innervation post implantation to ensure functional recovery of repaired tissue.

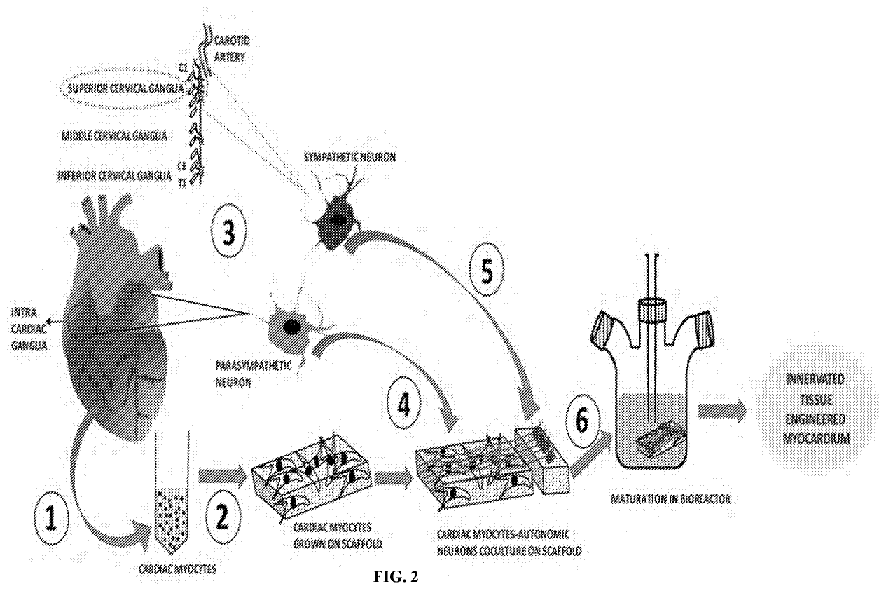

In order to fabricate innervated tissue engineered myocardium it is necessary to select the appropriate cell types, scaffolds, and culture conditions for construct maturation. Embodiments comprising several of these criteria are illustrated in FIG. 2. Cardiac myocytes, the principal cell type in the heart can be isolated from embryonic or neonatal myocardium following established protocols and cultured on scaffolds for 3-5 days before addition of autonomic neurons for co-culture to obtain innervated cardiac tissue. The choice of scaffold will be determined by the end application of the construct (e.g., injectable hydrogel, myocardial patch or a whole bioengineered heart). The intracardiac ganglia appear to be the appropriate choice for parasympathetic neurons while the sympathetic population can be harvested from superior cervical ganglia. To mimic the anatomy of axons travelling to distant organs from the ganglia the ganglia are cultured on a separate scaffold placed at a distance. Since the postganglionic parasympathetic pathway is embedded with the atrium (cardiac ganglia) of the heart, only the sympathetic ganglia may be cultured on a separate scaffold until there is noticeable axonal extension into the nearby scaffold containing cardiac myocytes and parasympathetic neurons (as shown in FIG. 2). Subsequently, the construct may be allowed to mature in a bioreactor (microfluidic/perfusion batch) to form an innervated cardiac tissue.

### Tissue Engineered Innervated Pancreatic Tissue

In another aspect, the invention provides a method of generating innervated tissue engineered pancreatic tissue by isolating pancreatic acinar and beta islet cells; culturing the pancreatic acinar cells and beta islet cells on a first scaffold; isolating and culturing sympathetic ganglia and parasympathetic neurons; co-culturing parasympathetic neurons with the pancreatic acinar cells and beta islet cells on the first scaffold; culturing the sympathetic ganglia on a second scaffold adjacent to the first scaffold; and maturing the construct in a bioreactor; thereby generating innervated pancreatic tissue.

Parasympathetic preganglionic fibers emanate from the vagus, innervating the pancreas along the vasculature and synapsing with postganglionic fibers in intrapancreatic ganglia. In the case of sympathetic innervation, the cell bodies of preganglionic fibers emanate from the hypothalamus and reach the celiac ganglia from where postganglionic fibers extend to innervate the pancreas following blood vessels. The innervation pattern in the pancreas appears to be species-specific. Mouse pancreatic islets exhibit dense parasympathetic and sympathetic innervation in direct contact mainly with α-/β-cells and α-cells, respectively, in significantly greater amounts than innervation in the exocrine pancreas. On the other hand, more recent investigations have determined that human islets are scarcely innervated, with most of the fibers likely being of sympathetic origin as determined by immunohistochemical staining of human pancreatic sections. The majority of sympathetic axons do not associate directly with endocrine cells; instead, fibers have been found parallel to islet blood vessels and innervating SMCs.

Pancreatic development initiates at E9.5 in mice with the formation of buds emerging from the foregut endoderm that subsequently merge. This is followed by an extensive branching of the pancreatic epithelium to form a tubular network and the delamination of endocrine precursors and their aggregation into clusters that will constitute the pancreatic islets. Sympathetic neurons, identified by vesicular monoamine transporter 2 (VMAT2) staining, have been observed as early as E12.5 in the pancreatic bud of embryonic mice. The VMAT+ fibers acquire their adult configuration and associate with blood vessels along with islet maturation in the postnatal stage.

Pancreatic sympathetic innervation appears crucial for determining the final islet cytoarchitecture during development. It has been postulated that the adult islet structure is fundamental for proper interactions between β-cells and for insulin secretion, with alterations related to diabetes in humans. Denervation with 6-OHDA in neonatal mice resulted in a loss of the globular, clustered cytoarchitecture of α-cells surrounding a β-cell core. Similarly, mice denervated by genetic ablation of the NGF receptor TrkA in sympathetic neurons presented disorganized islets with α- and β-cells lacking the necessary intercellular contacts. The TrkA receptor is a suitable target because NGF is required for the survival and targeting of innervating neurons. Postnatal TrkA mutants, which had a complete loss of sympathetic innervation in the pancreas, also displayed islets with reduced expression of neural cell adhesion molecule (N-CAM) and E-cadherin and in greater proximity to pancreatic ducts, suggesting that innervation is necessary for proper islet cell-cell adhesion/clustering and migration. Furthermore, innervation also influences the functional maturation attained by islets during development, as islets of mutant 1 month-old mice exhibited reduced glucose-stimulated insulin secretion in vitro, decreased expression and surface localization of the Glut2 glucose transporter, and reduced docking of insulin granules at the plasma membrane.

Even though the pancreas can function independent of innervation, the ANS has been implicated in the cephalic phase of insulin release, preservation of normal glucose tolerance after food ingestion, synchronization between pancreatic islets, response to glycopenic stress, and in development of diabetes in adulthood when dysfunctional. Traditionally parasympathetic innervation has been considered to promote insulin release through muscarinic receptor activation in β-cells, as shown by experiments with exogenous agonists and VNS in several species including humans. Moreover, specific hypoglycemia levels activate parasympathetic innervation to also modulate glucagon secretion. Sympathetic innervation stimulation inhibits glucose-stimulated insulin secretion and promotes glucagon release due to interactions between NE and adrenergic receptors on islet cells. Due to the differences found between the type of contacts between autonomic fibers and pancreatic islets, two possible mechanisms of functional regulation have been suggested. Direct neurotransmitter release onto the innervated islet cells is the probable means by which neurons exert control over pancreatic hormone release in mice. On the other hand, in humans it has been proposed that sympathetic fibers may accomplish this control by causing contraction of proximal islet blood vessels due to NE release, thus regulating blood flow in the islet. In addition, NE could be carried by vessels to perfused islets to act on adrenergic receptors in what is called the “spillover” mechanism. More knowledge is required in this field, particularly for parasympathetic innervation since the spillover explanation is not consistent with ACh being rapidly degraded by acetylcholinesterases. As with the heart, studies have suggested a relationship between innervation and pancreatic disease. In rats with early diabetes (1-2 weeks), a significant decrease in sympathetic fibers in the endocrine pancreas has been reported. Non-obese diabetic mice with insulitis showed reductions in islet innervation, accompanied by increased expression of neurotrophins that suggested the need to promote nerve ingrowth.

Electrical stimulation of splanchnic nerves and the spinal cord has been used for management of pain arising from chronic pancreatitis in humans. The secretory function of pancreas can be modulated through specific stimulation of the sympathetic and parasympathetic branches near the organ. Stimulation of sympathetic nerves near the pancreatic artery inhibits secretion of somatostatin, pancreatic polypeptide and insulin with a rise in glucagon levels. VNS leads to postganglionic fibers promoting the release of insulin from beta cells by increasing the cytosolic concentration of Ca²⁺ from intracellular reservoirs, mediated by the inositol trisphosphate (IP3) receptor, to promote exocytosis of insulin granules. VNS also promotes release of glucagon, somatostatin, and pancreatic polypeptide from alpha, delta, and gamma cells in the islets, respectively.

The effect of loss of sympathetic innervation on pancreatic pathophysiology has been studied in a variety of animal models like insulin resistant, type 2 diabetic and non-obese diabetic models. However, tissue engineering efforts to regenerate pancreatic function have focused more on the importance of vascularization of the artificial construct and have considered sympathetic innervation as a mere byproduct of revascularization. Interestingly, pre-vascularized pancreatic constructs have been reported to exhibit delayed onset of function following both whole pancreas and islet cell transplantation due to sporadic sympathetic innervation. The crucial role of innervation and neurochemical cues in development and function of the pancreas makes it an essential prerequisite in developing an artificial pancreatic construct that has high biological fidelity and functionality.

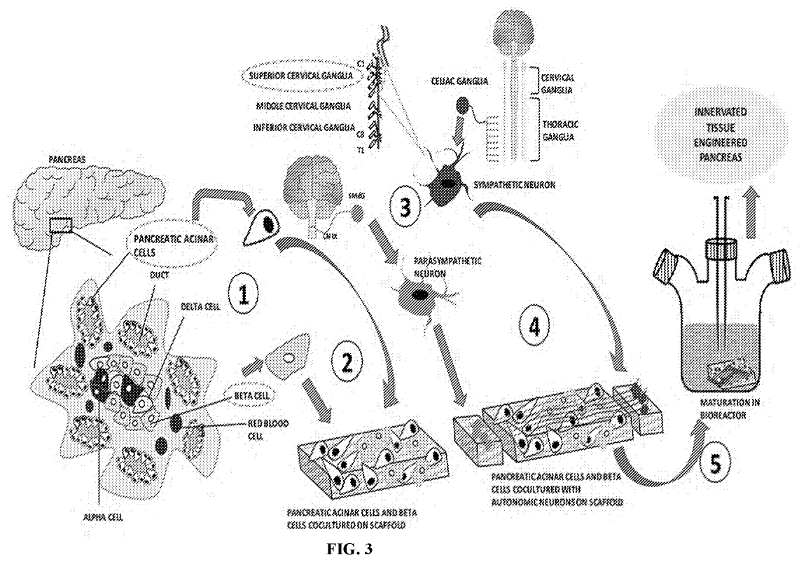

Based on the physiology of the pancreas, in order to engineer a functional pancreatic tissue it is necessary to co-culture pancreatic acinar cells and beta islet cells on an appropriate scaffold as described in FIG. 3. Beta islet cells can be isolated and cultured from rat or human tissue as described in previous literature. Beta cells have been reported to have a strong preference for specific extracellular matrix (ECM) components which should be considered for designing the scaffold. Primary pancreatic acinar cells from mice or human can also be isolated following established protocols. The pancreas receives preganglionic parasympathetic input from the vagus nerve and postganglionic input from the intrapancreatic ganglia. It is highly challenging to access the dorsal motor nucleus of the vagus and there appear to be no reports about isolation and primary culture of cells from intrapancreatic ganglia. Hence, the submandibular ganglia is the most appropriate choice for harvesting parasympathetic populations. Sympathetic neurons can be harvested from the well-established but anatomically distant superior cervical ganglia or from the celiac ganglia which is more anatomically relevant but less studied in terms of primary cell culture. Since islet cells have been reported to mature better in the presence of neurons, simultaneous co-culture of the islets and autonomic neurons provides the appropriate milieu for their growth. The parasympathetic and sympathetic populations are seeded on separate scaffolds as described in FIG. 3 to recapitulate the native anatomy of long axons projecting from autonomic ganglia into the pancreas. Once the scaffold containing the co-culture is adequately innervated with parasympathetic and sympathetic axons, the entire setup can be transferred into a bioreactor for maturation leading to the formation of an innervated pancreatic tissue.

### Tissue Engineered Innervated Intestinal Tissue

In another aspect the invention provides a method of generating innervated intestinal tissue by isolating intestinal smooth muscle cells; culturing the intestinal smooth muscle cells on a first scaffold; isolating and culturing enteric neurons; co-culturing the enteric neurons with the intestinal smooth muscle cells on the first scaffold; and maturing the construct in a bioreactor; thereby generating innervated intestinal tissue.

The gastrointestinal (GI) tract is extrinsically innervated by parasympathetic fibers from the vagus that end up throughout the entire tract, with less abundance in the colon and distal small intestine, while the postganglionic sympathetic innervation emanates from prevertebral ganglia. The enteric nervous system (ENS) is the intrinsic nervous system component of the GI tract that innervates components of the gut wall with approximately 200-600 million neurons. The ENS is formed by a myriad of ganglionic plexuses (i.e., myenteric and submucosal plexus) that regulate motility, secretion, and blood flow. The myenteric plexus is a continuous circuit covering the entire GI tract, while the submucosal plexus is mainly observed in the small and large intestine. The neural content of the GI tract is completed by the presence of intestinofugal neurons, with somata in the ENS ganglia that synapse onto sympathetic ganglia to regulate luminal transit in the stomach and proximal/distal small intestine as part of the entero-enteric reflexes. The extrinsic innervation is not essential for GI function, as evidenced by several studies that showed non-significant morbidity effects after vagotomy and sympathectomy procedures. Intrinsic ENS innervation can independently control GI function, and its importance is markedly shown through a variety of pathologies resulting from enteric neuropathies.

The development of the ENS occurs near its end targets, contrary to how this process happens in the CNS. Interestingly, migration of enteric neural crest cells (NCCs), smooth muscle cell and interstitial cell of Cajal (ICC) differentiation, and mucosa development all proceed in a rostral-to-caudal direction. Common signaling pathways (e.g., Sonic hedgehog, bone morphogenetic protein, platelet-derived growth factor) influence aspects of both ENS and gut development such as patterning, villi/crypt formation, stem cell and enteric neuron/glia proliferation and differentiation, cell cycle timing, neuron migration, and neurite fasciculation and directionality. In mice, vagal NCC-derived ENS precursors reach the foregut by E9.5 and migrate in a rostral-to-caudal manner to colonize the myenteric region; by E12.5 synaptic vesicles appear in the stomach; by E14.5 Schwann cell precursors migrate into the gut and neurites grow into the circular muscle. Neurites extend into the mucosa by E16.5, and during P0-P7 enteric glia enters it. Particularly, neuronal control over motor complexes in the mouse duodenum and colon occur by E18.5 and P8-P14, respectively.

The extrinsic and intrinsic innervation of the digestive system is involved in the regulation of bowel transit, smooth muscle contraction, gastric volume, acid, hormone and enzyme secretion, local blood flow, nutrient absorption, and expulsion of pathogens and harmful substances. Innervating fibers in the gut also interact extensively with the intrinsic GI immune and endocrine system. The relative importance of the intrinsic and extrinsic components of GI innervation in the regulation of motility depend on the organ. Vagal pathways play a more prominent role in controlling muscle activity in the esophagus and stomach, while the ENS is more essential for this function in the small and large intestines with the exception of the rectum. Intrinsic and extrinsic neurons are integrated, particularly by sympathetic pathways and enteric secretomotor/vasodilator neurons, to maintain local and body fluid and electrolyte balance.

Vagal sensory neurons serve as mucosal chemoreceptors, mechanoreceptors, and stretch receptors that respond to stimulation in the lumen mainly in the esophagus, stomach, and proximal small intestine. Vagal efferents mostly connect with enteric circuits in the esophageal smooth muscle and lower sphincter, stomach, gallbladder, and pancreas. Postganglionic sympathetic neurons provide inhibitory input to the submucosal and myenteric plexi to constrain secretomotor activity and GI transit, respectively. The majority of sympathetic innervation in muscle regions is found in sphincters, similarly inhibiting the passage of luminal contents. In addition, vasoconstrictor neurons from paravertebral and prevertebral ganglia innervate and constrict intramural arteries in the gut wall. Pelvic nerves provide afferent innervation that relays information from mechanoreceptors that are sensitive, for example, to pain in the distal GI tract. Efferents from the pelvic and lumbosacral ganglia act as pre-enteric neurons in the distal colon and rectum or as direct innervating fibers to the colon, participating in vasodilation, propulsion, and defecation.

On the other hand, among ENS neurons, there are the multi-axonal intrinsic sensory neurons, found in the myenteric and submucosal plexi that are sensitive to mechanical distortion and the chemistry of luminal contents. Uni-axonal excitatory and inhibitory neurons innervate the two muscle layers and the muscularis mucosae to modulate smooth muscle contraction and relaxation by the secretion of excitatory (e.g., ACh, tachykinins) and inhibitory (e.g., nitric oxide, vasoactive intestinal peptide) neurotransmitters, respectively. The mucosa is populated by secretomotor and secretomotor/vasodilator neurons that promote exocrine fluid secretion and increased blood flow in cholinergic and non-cholinergic varieties. Various types of ascending and descending interneurons in the ENS participate in motility and secretomotor reflexes, as well as in migrating myoelectric complexes.

Modulation of GI activity has been reported using temporary as well as permanent gastric electrical stimulation devices for both upper and lower GI tract disorders. Temporary endoscopic placement of these devices and subsequent stimulation resulted in the mitigation of various upper GI disorder symptoms such as vomiting and nausea. Upper GI stimulation mainly works by modulating the ENS and is considered safe for long-term use. In cases of lower GI disorders like fecal incontinence and constipation, sacral nerve stimulation (SNS) has emerged as a viable treatment method. Being a minimally invasive technique and with reported success in two-third of cases. SNS has been extended to patients with sphincter disruption, evacuation difficulty, neurogenic bowel dysfunction, and recently even irritable bowel syndrome (IBS).

Innervated smooth muscle sheets are fabricated in vitro by coating aligned smooth muscle cells with enteric neural progenitors, and these tissues may be stimulated electrically and chemically and exhibited muscle and neuron-dependent contraction and relaxation. Human intestinal organoids have been mechanically aggregated with human NCC-derived ENS precursors prior to culture in 3D conditions and maturation in vivo. The contractile capacity of these grafts mimicked human intestinal motility only when it incorporated ENS neurons, showing again the paramount importance of innervation to achieve native-like functionality. Moreover, the fact that the ENS plays an integral role throughout the prenatal stage of GI development indicates that the presence of ENS neurons (or NCC-derived ENS precursors) is essential for successful fabrication of matured engineered GI tissues.

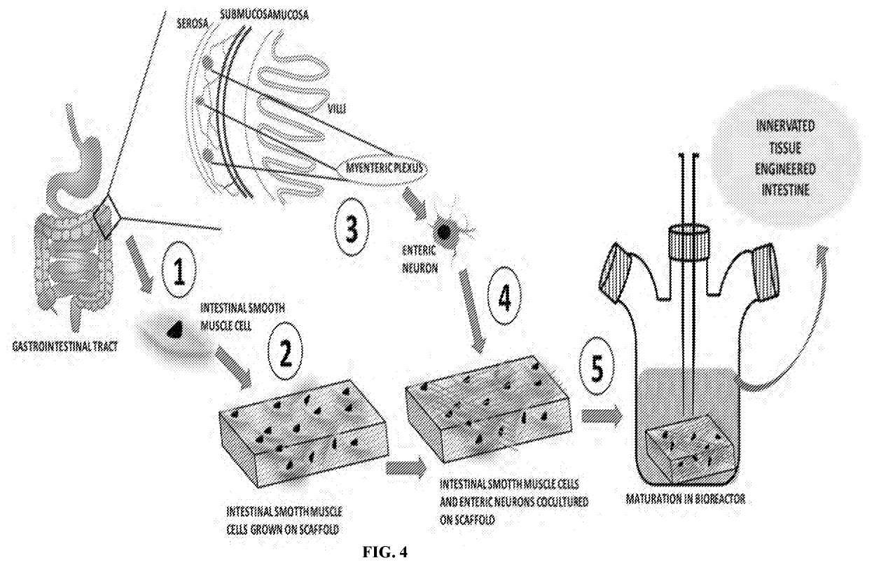

FIG. 4 illustrates an embodiment of the invention by which innervated intestinal tissue is engineered. Intestinal smooth muscle cells (SMC) may be harvested from murine or human tissue and co-cultured with enteric neurons derived from precursor cells or from primary culture of the myenteric plexus. Simultaneous co-culture of intestinal SMCs and enteric neurons is the best strategy. The cells are cultured on 3D scaffolds and allowed to mature in a bioreactor.

### Tissue Engineered Innervated Salivary Gland Tissue

In another embodiment, the invention provides a method of generating innervated salivary gland tissue by isolating salivary acinar cells; culturing the salivary acinar cells on a first scaffold; isolating and culturing sympathetic and parasympathetic neurons; culturing sympathetic neurons on a second scaffold, culturing parasympathetic neurons on a third scaffold, wherein the second scaffold and the third scaffold are adjacent to the first scaffold; maturing the construct in a bioreactor; thereby generating innervated salivary gland tissue.

The parotid gland is innervated by postganglionic parasympathetic nerves from the otic ganglion near the base of the skull that synapse with preganglionic fibers from the inferior salivatory nucleus in the medulla. Moreover, preganglionic nerves from the superior salivatory nucleus in the pons join the facial nerve and then the lingual nerve to connect with postganglionic parasympathetic neurons in the submandibular ganglion, from which the submandibular and sublingual glands are innervated. The sympathetic pathway consists of preganglionic fibers that synapse at the superior cervical ganglia from which postganglionic fibers innervate the salivary glands through the external carotid plexus.

Studies with the mouse submandibular gland have shown that by E11 the oral epithelium inserts itself into neural crest-derived mesenchyme, grows a single epithelial duct by E12, and produces a highly-branched gland by E14. In vitro and in vivo studies have demonstrated that the early parasympathetic innervation from the submandibular ganglion develops in conjunction with epithelial morphogenesis of the submandibular gland, with axons following the path dictated by the branching pattern. Moreover, branching of the initial epithelial bud in the developing parotid gland commences once postganglionic nerves from the otic ganglion reach it. Parasympathetic innervation also maintains undifferentiated epithelial progenitor cells needed for the formation of the salivary gland. Removal of the parasympathetic submandibular ganglion in mice resulted in a reduced presence of epithelial progenitors in the embryonic gland, evidenced by a lesser expression of the progenitor markers cytokeratin-5 and cytokeratin-15, as well as decreased end buds during development in explant culture. This effect was replicated by the use of ACh and muscarinic receptor 1 signaling inhibitors and rescued by the application of ACh analogs, demonstrating the need for ACh typically provided by parasympathetic innervation. Furthermore, this innervation has been established as a regulator of tubulogenesis. Curtailed parasympathetic innervation by blocking Neurturin signaling impedes ductal tubulogenesis. Vasoactive intestinal peptide was identified as the neurotransmitter responsible for promoting ductal growth and lumen formation. Resection of the chorda tympani nerve, which carries preganglionic parasympathetic fibers in their journey to the submandibular ganglion, within a critical time window of 48 hour after birth resulted in the inhibition of differentiation or bundling of myoepithelial cells in acinar buds even at 60 days after birth. Nevertheless, parasympathectomy at later time points did not affect the normal acinar bud maturation observed in intact glands, suggesting an influence mainly in early postnatal development. This procedure at several time points caused glands to weigh only around 60% of the weight of contralateral, innervated glands at 60 days. Similarly, sympathectomy by severing the superior cervical ganglion 4 hour after birth led to buds with significantly lower acinar cell size and granule content postnatally after 9 weeks. A similar surgical procedure performed on adult rats had the effect of significant reductions in the size of the parotid gland relative to the contralateral control and alterations in the production of parotid proteins (e.g., proline-rich proteins, deoxyribonuclease) even after 12 weeks.

Parasympathetic and sympathetic nerves regulate the secretion of saliva from major and minor salivary glands in various degrees, a process required for proper lubrication, digestion, immunity and homeostasis, promote contraction of myoepithelial cells, and regulate blood flow in the glands. Parasympathetic and sympathetic postganglionic fibers mainly exert their effects through ACh and NE, respectively, but other neurotransmitters are also utilized. Parasympathetic inputs evoke most of the secretion of saliva, particularly that of serous-watery, serous-mucous, and mucous saliva from the parotid, submandibular, and sublingual glands, respectively. On the other hand, sympathetic innervation has been considered to be important for promoting exocytosis of proteins from granules in acinar cells, but parasympathetic stimulation can also play a role in this process. Removal of the sympathetic source of innervation has also demonstrated that these fibers influence the control of inflammatory and immune mediators in salivary glands.

Electrical stimulation near the chorda lingual nerve (CLN), glossopharyngeal and vagus nerve modulated parasympathetic salivary secretion and vasodilation from the parotid and submandibular glands in sympathectomized cats. Neuroelectrostimulation through external or implantable electronic devices called “salivary pacemakers” are widely used in patients with xerostomia. First generation salivary pacemakers like Salitron were comprised of a probe to be placed in between the tongue and the palate for delivering electrical stimuli to the sensory neurons and induce salivation. It showed promising results in preliminary clinical studies on patients with Sjögren's syndrome, which led to the development of further advanced technologies. Second generation devices (developed by GenNarino Saliwell Ltd. Germany) were removable intraoral appliances and did not produce any adverse local or systemic effect. Third generation “salivary pacemakers” by Saliwell were osteointegrated implants placed near the lingual nerve. These devices can generate continuous or frequent stimuli to keep the oral cavity moist and can be operated in “autoregulatory mode” as well as by the patients via remote control.

Salivary gland damage can result from radiation therapy, aging and Sjogren's syndrome. The most prominent form of salivary gland impairment is xerostomia (dry mouth symptom) which can lead to multiple pathologies related to dental and oral health including bacterial infection, swallowing dysfunction and dysgeusia (lack of taste). Orthotopic transplantation of a bioengineered salivary gland (submandibular) developed from epithelial and mesenchymal germ cell layers in adult mice led to innervation of graft by 30 days in vivo. Such innervation into the bioengineered construct allowed induction of salivary secretions by stimulating the parasympathetic pathways using pilocarpine as well as by citrate mediated gustatory stimulation. Hence, it is essential for an artificial salivary gland to have pre-existing neural networks or promote innervation to ensure proper maturation and functioning.

Engineering a functional salivary gland would first require selecting the appropriate source of salivary acinar cells depending on the target gland (parotid/submandibular/sublingual). Once the cells are harvested, they are maintained in primary culture until maturation and followed by subsequent co-culture with autonomic neurons on scaffolds as described in the FIG. 5. The parasympathetic and sympathetic populations are isolated from the submandibular and superior cervical ganglia respectively and cultured on separate scaffolds. Salivary epithelial cells and cortical neurons were simultaneously plated in a previously reported co-culture model. Hence, simultaneous co-culture of acinar and autonomic neurons is the best model to follow. Following axonal infiltration from both parasympathetic and sympathetic populations into the salivary acinar cells, the entire setup is allowed to mature in a bioreactor to form an innervated salivary gland.

### Tissue Engineered Innervated Skeletal Muscle Tissue

In another aspect, the invention provides a method of generating innervated skeletal muscle tissue by isolating skeletal myocytes; culturing the skeletal myocytes on a first scaffold to form myofibers; isolating spinal motor neurons; co-culturing the motor neurons with the myofibers on the first scaffold; maturing the construct in a bioreactor; thereby generating innervated skeletal muscle tissue. In various embodiments, the method further comprises forced aggregation of the spinal motor neurons prior to co-culture on the first scaffold.

The musculoskeletal system is mostly under the voluntary control of the somatic nervous system. The basic unit of contraction in the skeletal muscle is the motor unit, composed of a somatic motor neuron that innervates multiple myofibers. Somatic motor neurons are classified into alpha, beta and gamma type depending on the type of muscle fiber they innervate. All three types have their cell bodies in the ventral horn of the spinal cord. Alpha motor neurons innervate the extrafusal muscle fibers (fast-twitch) that primarily produce fast higher energy for shorter periods of time. The intrafusal muscle fibers (slow-twitch), that act as proprioceptors for stretch, are innervated by the gamma and beta motor neurons and produce less energy but for longer periods. Nerve activity is a major control mechanism of the fiber type profile. Interestingly, increased neuromuscular activity and mechanical loading induces transition of fast-to-slow, while reduced neuromuscular activity and mechanical loading causes transitions in the slow-to-fast direction. Moreover, denervation and immobilization induce preferentially fast-type fiber atrophy, while cachexia (wasting syndrome) and chronic heart failure induces slow fiber atrophy. An important question in neuromuscular biology is how skeletal muscles interpret the stimulation coming from motor neurons to define their phenotype as slow or fast fibers. Clearly, understanding how neuromuscular activity alters fiber-type transitions could lead to stronger skeletal muscle formation.

The development of skeletal muscle or myogenesis initiates from progenitor cells originated from the somites. These cells delaminate from the hypaxial edge of the dorsal part of the somite, called the dermomyotome, and migrate into the limb bud, where they proliferate, express myogenic determination factors and subsequently differentiate into skeletal muscle. Myogenesis is divided into primary myogenesis (embryonic stage) when primary muscle fibers arise and secondary myogenesis (fetal stage), that leads to the formation of secondary muscle fibers. The myogenic differentiation of the committed cells, termed myogenic progenitors, and subsequent formation of myoblasts is under control of a variety of growth factors, transcription factors and neurotrophins. Once the secondary myofibers have formed, they begin to grow by the continued fusion of fetal myoblasts and later on the formation of the neuromuscular junction, going from multi-innervation to a single innervation of each myofiber to ensure the skeletal muscle functionality.

Skeletal muscles express a number of neurotrophic receptors implying the crucial role played by such molecules in development and maintenance of muscle architecture and function. For example, neurotrophin 3 and NT 4/5 are necessary for normal muscle development and their deficiencies can lead to slow muscle fiber degeneration and loss of proprioception. In the rodent pre-natal stage, innervation into myotubes starts around E15 and is mainly polyneuronal in manner i.e. each motor endplate is served by multiple motor neurons. Within the first two weeks of birth in rodents, there is extensive pruning of synapses and retraction of nerve fibers through a process called “synapse elimination” that ultimately leads to mono-neuronal architecture of motor endplate (single motor neuron connects to a single motor endplate). The sequence of events is similar in humans albeit with a delay. In rodents, synapse elimination is further accompanied by “switch” of acetylcholine receptors (AChR) expressed in myotubes from gamma (fetal) to epsilon (adult) type. In humans, this “switch” occurs at least 6 weeks after synapse elimination. Although the diameter of muscle fibers and muscle volume increase multiple times with postnatal muscle growth and/or exercise, the number of presynaptic neuromuscular apparatus remains the same as the first year of birth.

The neural input for muscle contraction originates in the primary cortex region (first order motor neurons) and travels through the corticospinal tracts to reach the ventral horn of the spinal cord that houses the second order motor neurons. The signal is transmitted from the nerve terminal to specific myofibers by secretion of neurotransmitters at the neuromuscular junctions (NMJ). This leads to depolarization of muscle fibers through Ca²⁺ influx and generation of muscle action potential. The importance of innervation in skeletal muscle maintenance and function is highlighted by the debilitating and fatal consequences of various neuromuscular diseases. Autoantibodies against acetylcholine receptors (AChR) and voltage-gated calcium channels (VGCC) blocks post-synaptic and presynaptic functions at NMJs leading to acquired Myasthenia Gravis and Lambert Eaton Myasthenic Syndrome. Motor neuron degeneration in the brain and/or spinal cord can lead to Amyotrophic lateral sclerosis (ALS) characterized by stiffness and atrophy of skeletal muscles. Moreover, neuromuscular pathologies are present in other muscle diseases, including Duchenne and Becker muscular dystrophies and other motor neuron diseases such as progressive bulbar palsy and pseudobulbar patsy. Interestingly, disuse-induced skeletal muscle atrophy has been also associated with neuromuscular junction instability.

Extensive structural and functional damage to skeletal muscles can also be caused from physical trauma, chronic denervation or surgery and is referred to as volumetric muscle loss (VML). Free functional muscle transfer (FFMT) is the preferred procedure to treat VML that entails transplantation of donor muscle along with nerve and blood vessels from any part of the body to the injury site to facilitate re-innervation and re-vascularization of the graft region. Although FFMT remains the gold standard, its success is limited by donor site morbidity, long operative time and prolonged re-innervation of motor end plates in the donor muscle.

The motor neuron axon, in response to an action potential, releases a neurotransmitter in the post-synaptic membrane of the muscle fiber, which converts the chemical to a mechanical signal in the form of muscle contraction. When the dialogue between these compartments is compromised, as happens in chronic phasic electrical stimulation of a specific muscle, the contractile properties are altered significantly. Specifically, fast skeletal muscle fibers were found to be transformed into fast, fatigue resistant type upon continuous electrical stimulation in rabbit, porcine and humans. Neuromuscular stimulation using external stimulators have been used in sports medicine to increase isometric muscle strength, muscle mass and oxidative capacity of muscle following reconstructive surgery or injury. Similar technique has shown to facilitate rehabilitation/mobilization among intensive care patients suffering from muscle weakness and atrophy acquired due to prolonged periods of immobilization.

Tissue engineered skeletal muscle constructs are fabricated using scaffold based as well as scaffold-less technologies. Synthetic polymers as well as ECM proteins like collagen are used as scaffolds, whereas scaffold-free techniques involve self-assembly of skeletal muscle constructs using muscle stem cells (also called satellite cells) or tendon constructs to form 3D structures. Appropriate somato-motor innervations remain the biggest challenge to fabricating a fully functional muscle. Engineered muscle constructs developed using self-assembly of primary myocytes have been reported to interface with surrounding neural tissue in vivo when surgically connected to the sural nerve. 3D printed skeletal muscle tissue grafts surgically embedded with common peroneal nerve led to the formation of NMJs 2-week post implantation. In order to promote innervation, muscle grafts have been surgically inundated with multiple surrounding nerves (hyper-innervation). In another approach, embryonic motor neurons were injected into the distal tibial nerve stump one week after a sciatic nerve transection. Regenerating axons were found to be myelinated and of smaller diameter forming simple NMJs. Intramuscular axon sprouting from transplanted neurons augmented muscle reinnervation, reduced atrophy and restored muscle excitability. Innervation plays a crucial role in development, maturation and functional regulation of the musculoskeletal system and hence it is imperative that a tissue-engineered muscle be pre-innervated during construction and capable of robust innervation upon implantation.

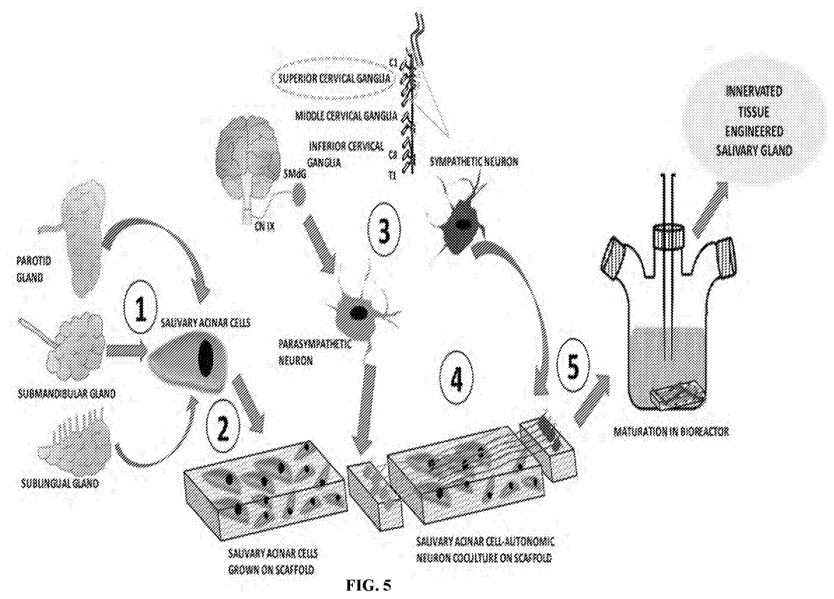

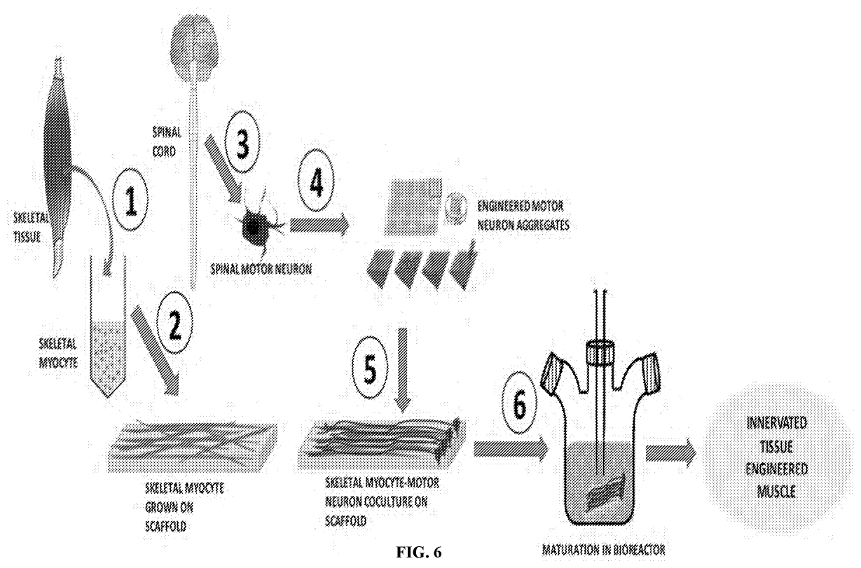

In cases with significant neuromuscular damage/loss, the ideal surgical intervention would entail fabrication and implantation of bioengineered nerve-muscle complexes, however such has yet to be developed. There are numerous reports describing fabrication of neuromuscular junctions in vitro through co-culture of motor neuron and skeletal myocytes. However, such co-cultures do not have sufficient biomass to make them implantable for repair/replacement of injured neuromuscular tissue. The invention provides a method to generate engineered aggregates of spinal motor neurons that can project long aligned axons and withstand mechanical forces. In order to generate an implantable neuromuscular construct, engineered motor neuron aggregates are cultured on a bed of pre-differentiated myofibers grown on a suitable substrate (FIG. 6). This entails culturing the skeletal muscle cells for 4-7 days in differentiation media to allow formation of myofibers prior to addition of motor neurons. Following formation of neuromuscular connections, the construct may be allowed to mature in a bioreactor to form an “off-the-shelf” implantable nerve-muscle complex.

### Tissue Engineered Innervated Spleen Tissue

In another aspect the invention provides a method of generating innervated spleen tissue by isolating sympathetic neurons; culturing the sympathetic neurons on a first scaffold while allowing axonal growth to an adjacent second scaffold; isolating splenocytes; co-culturing the splenocytes on the first scaffold with the sympathetic neurons; thereby generating innervated spleen tissue.

The spleen is innervated by postganglionic sympathetic axons through the splenic nerve, which have cell bodies in the prevertebral sympathetic ganglia and create a network that follows the branches of the splenic artery mainly to the white pulp region and occasionally the marginal zones. These axons interface with preganglionic fibers within the greater splanchnic nerve coming from thoracic spinal cord, predominantly from T9-T11. The spleen appears to lack parasympathetic innervation although there have been conflicting reports on this matter. A study with anterograde tracing of the dorsal motor nucleus showed that branches of the vagus provide efferents to the celiac, mesenteric, and suprarenal ganglia, but more recent studies failed to find putative synaptic connections between vagal efferents and sympathetic neurons in the ganglia.

Studies in the rat spleen showed noradrenergic fibers in the white pulp at birth, which grow continually at pace with the spleen compartments. These fibers surround growing primary follicles or condense within the Periarteriolar lymphoid sheaths (PALS) as T lymphocytes are redistributed to the inner PALS and B lymphocytes are excluded to the outer region and the marginal zone.

Splenic innervation is intimately involved in the regulation of the innate immunity involved in the first line of defense against immune challenges through the actions of natural killer cells mast cells, dendritic cells, macrophages, neutrophils, among other types of leukocytes. Inflammatory cytokines in this acute response, such as tumor necrosis factor-alpha (TNF-α), are involved in changes in gene expression and vascular permeability, increases in the recruitment of immune cells to infected sites, the death of damaged cells, the production of fever, and antigen presentation for the adaptive response. Exposure to an immune insult such as lipopolysaccharide (LPS) endotoxin has been shown to increase the activity of the splenic nerve, which mediates these immune responses mainly through NE released from its postganglionic sympathetic axons. Studies have elucidated that NE binds mainly to β-adrenergic receptors on the surface of splenic macrophages to downregulate the production of TNF-α when exposed to LPS. Exposure to stressors can also stimulate the SNS to reduce the innate immune response, and resection of the splenic nerve has been shown to prevent stress from reducing inflammatory cytokine levels in endotoxemic models.

Due to the functional relationship between splenic function and its innervation, neurological damage can result in impaired immune function. For example, high-level spinal cord injury can weaken or ablate the input to preganglionic fibers from the spinal cord, leading to elevated levels of NE in the spleen, increased splenocyte apoptosis, dysregulated antibody synthesis in a β2 receptor-dependent manner, and overall increased susceptibility to infection in animal models and human patients dependent on the level of injury on the preganglionic neurons. Recent studies elucidated that this immune-suppression is related to chronic maladaptive plasticity below the injury that forms an intraspinal anti-inflammatory reflex circuit that becomes hyperactivated when spinal interneurons are spontaneously stimulated. The excessively secreted NE can also suppress osteoblast function, which may also account for the osteoporosis typically exhibited by SCI patients.

Severe sepsis is the major cause of death in non-coronary intensive care units, and rheumatoid arthritis (RA) and inflammatory bowel diseases are suffered approximately by 1.3 and 1.4 million Americans, respectively. Typical drug treatments lack universal effectiveness and carry side effects related to immunosuppression and toxicity. These problems have led to the development of electrical devices that can stimulate nerves involved in the inflammatory reflex. Studies have shown that VNS reduces inflammatory cytokine levels and disease severity in animal models of sepsis, colitis, pancreatitis and arthritis, promotes remission in patients with Crohn's disease. Human patients with active RA have also been implanted with a vagus nerve stimulator on the cervical vagus nerve and subjected to a regimen of electrical stimulation over a 84 day period, resulting in significantly reduced TNF-α presence in the serum after stimulation and improvements in RA symptoms measured by clinical composite scores. Despite this, electrical stimulation is inherently non-specific and may cause off-target effects, while optogenetics mediated optical stimulation is limited by the need for opsin delivery methods that ensure sufficient/targeted expression and safety.

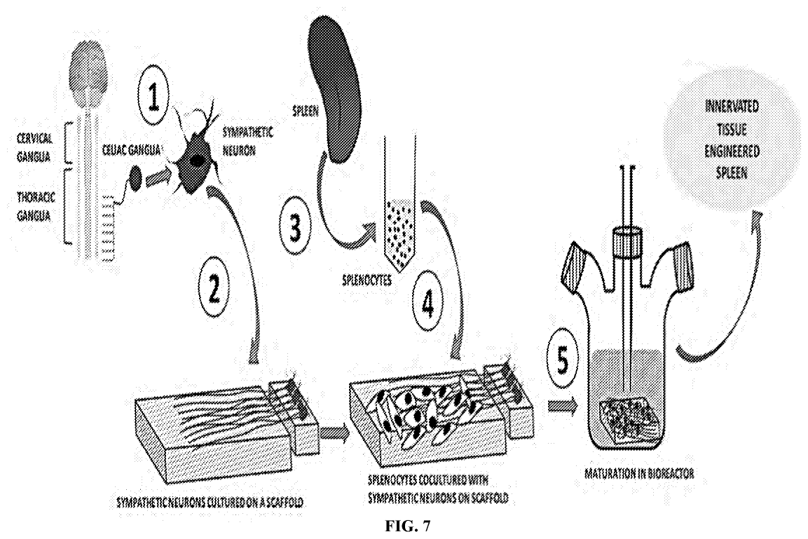

The spleen consists of a variety of white blood cells, dendritic cells and macrophages collectively referred to as splenocytes. Hence tissue engineering strategies involve isolation and primary culture of splenocytes as per established procedures to accurately recapitulate splenic physiology. FIG. 7 depicts an embodiment directed to a tissue engineering strategy to develop innervated splenic tissue. Sympathetic neurons may be harvested from the celiac ganglia and should be cultured for approximately over a week in vitro on a 3D scaffold before addition of splenocytes. Upon adequate innervation of the splenocytes, the construct may be allowed to mature and gain biomass by culturing it in a bioreactor. This can lead to an innervated and functional splenic tissue which can be used as an in vitro test bed or an implant to repair/replace damaged spleen.

### Tissue Engineered Innervated Bladder Tissue

In another aspect the invention provides a method of generating innervated bladder tissue by isolating bladder smooth muscle cells and urothelial cells; co-culturing the bladder smooth muscle cells and the urothelial cells on a first scaffold; isolating sympathetic neurons and parasympathetic neurons; co-culturing the sympathetic and parasympathetic neurons on separate scaffolds adjacent to the first scaffold; maturing the construct in a bioreactor; thereby generating innervated bladder tissue.

The urinary bladder is innervated by both the sympathetic and parasympathetic pathways of the autonomic as well as branches of somatic nervous system. Parasympathetic preganglionic neurons originate from the sacral segment (S2-S4) of the spinal cord and mostly innervate the detrusor muscle wall and the pelvic plexus with a small population present in the urothelium. The sympathetic pathway originates in the lower thoracic and upper lumbar spinal cord segments (T10-L2) and takes a complex route into the inferior mesenteric ganglia ending in the pelvic plexus via the hypogastric nerves. Somatic afferent neurons arising from the sacral dorsal root ganglia enter the pelvic and pudendal nerves whereas those from the rostral lumbar dorsal root ganglia innervate the hypogastric nerves.

Since most of the studies on bladder development has been limited to rodents, an understanding of the timing and role of innervation in the development of urinary bladder in human have been heretofore less well understood. Axons start to innervate the developing bladder walls in humans within 13 weeks of conception. Rodents achieve neural control of bladder function within a few days after birth. Studies with embryonic mice reveal that the axons can be differentiated into sympathetic, parasympathetic and sensory types by E14-18. Genetic models of bladder dysfunction also provide information on the impact of appropriate innervation in bladder function. Animal models with deletion of nicotinic acetylcholine receptor Chrna3, Chrnb2 and Chrna4 have shown to result in megacystis characterized by bladder enlargement and incontinence most likely driven by neuronal dysfunction. Further studies are necessary to better understand the role of innervation in bladder development and more specifically the role of different neurotransmitters in promoting synaptic targeting to appropriate bladder tissue.

The somatic and autonomic pathways innervating the urinary bladder are responsible for sensing bladder pressure and regulating contraction of bladder muscles for urination. Parasympathetic pathways comprising mainly of pudendal nerves secrete acetylcholine that binds with muscarinic receptors present in bladder smooth muscles leading to bladder contraction. The sympathetic pathways are stimulated with increase of bladder pressure resulting from accumulation of urine. NE released from the sympathetic chains arising from the inferior mesenteric ganglion leads to relaxation of bladder wall. The afferent fibers present in the pelvic, hypogastric and pudendal nerves monitor volume and pressure on the bladder walls. With increased accumulation of urine, the parasympathetic tone increases but the alpha-motor neurons arising from the ventral horn of the sacral spinal cord keeps the external sphincter closed thereby enabling voluntary control of urination. During urination, the alpha motor neurons are temporarily inhibited leading to opening of the external sphincter and passage of urine.

Urination or bladder voiding is a complex process regulated by coordinated responses between the autonomic and somatic nervous systems. Hence neurologic insult often results in a variety of urine storage and bladder voiding related complications termed as ‘neurogenic bladder’. Neuromodulation via electrical stimulation has been a well-established treatment option for patients with overactive or neurogenic bladder when conventional therapies fail. Sacral and percutaneous nerve stimulation are both FDA approved techniques for ameliorating lower urinary tract dysfunction. Sacral neuromodulation (SNM) uses mild electric impulses to stimulate the sacral nerve which is often referred to as the pacemaker of the bladder. SNM was first introduced back in 1979 that required surgical implantation of leads. The technique has evolved into a less invasive procedure with advanced lead fixation methods requiring minimal incision leading to FDA approval for use in patients with overactive bladder in 2002. As per American Spinal Injury Association, SNM was able to restore urinary continence in 80% of patients suffering from overactive bladder. Long duration treatment with SNM in patients suffering from stroke, Parkinson's disease, and multiple sclerosis resulted in continued success for 4 years. Percutaneous tibial nerve stimulation (PTNS) is less invasive compared to SNM and involves electrical stimulation of the posterior tibial nerve using a 34-gauge needle placed percutaneously near the ankle. In a clinical study, 18 individuals suffering from lower urinary tract dysfunction due to multiple-sclerosis were treated with PTNS for 3 months. During follow-up 89% patients reported subjective satisfaction with their bladder condition. In a larger study on 70 multiple sclerosis patients, 82.6% subjects reported improved urinary urgency following a 3-month PTNS therapy. Separate clinical studies on limited subjects suggest PTNS therapy can mitigate urinary incontinence symptoms in patients suffering from Parkinson's and ischemic stroke. Despite these clinical trials, the efficacy of PTNS in neuromodulation of bladder remains debatable due to the small sample size, non-standard treatment plans and poor descriptions of the extent and severity of neurologic disease.

Engineering a functional bladder requires culture of urothelial cells along with the bladder smooth muscle cells. Both the cell types can be harvested from different layers of the bladder as shown in FIG. 8. An underlying layer of bladder smooth muscle cells facilitates growth and maturation of urothelial cells. This is achieved by initial culture of bladder smooth muscle cells on a scaffold followed by plating of urothelial cells that mimics the native architecture of the bladder wall. Although the bladder has autonomic and somatic control the proposed tissue engineering strategy is restricted to the autonomic pathway considering it plays a more important role in the development of the organ. Although the pelvic ganglia has been considered parasympathetic, it has come under scrutiny with a recent study suggesting it could be sympathetic in nature. Both the controversial pelvic ganglia and well established celiac ganglia are possible choices for culture of sympathetic neurons. The submandibular ganglia provides an easily accessible, well established pool of parasympathetic ganglia. Based on the timing of innervation during development of bladder (within a few weeks of conception in humans), the autonomic neurons are introduced early (within 2-3 days) in culture with the bladder cells to ensure proper maturation of bladder tissue in vitro. The two autonomic populations are cultured separately such that axons from each population can infiltrate and connect with the urothelial-smooth muscle co-culture. Subsequent culture in an appropriate bioreactor helps in formation of a matured innervated bladder tissue.

### Living Scaffolds for Directed Innervation of Biofabricated Organs

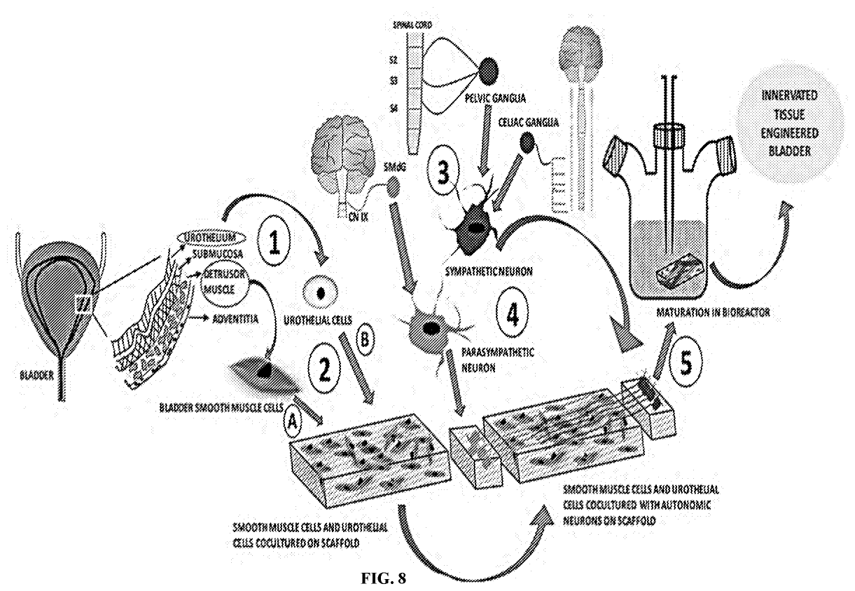

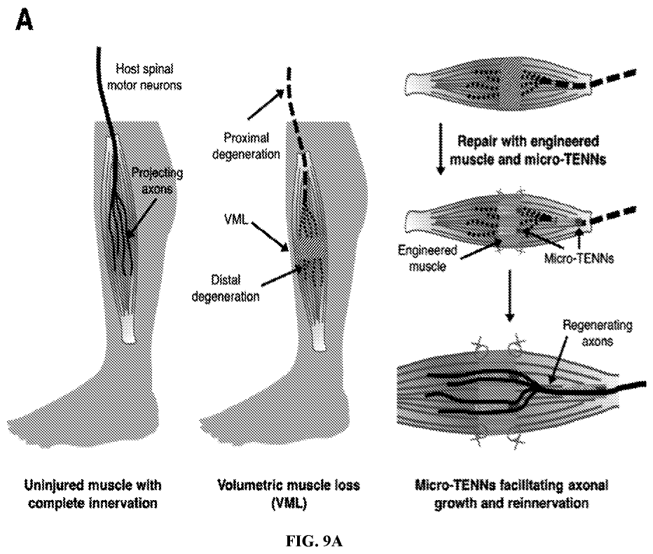

Neural tissue engineering involves combining biomaterial and cell-based strategies to augment nerve regeneration. “Living scaffolds” are neural networks consisting of phenotypically-controlled neural cells with a preformed 3D architecture often within a biomaterial matrix with controllable mechanical and biochemical properties. This architecture is precisely controlled to ensure that the structural composition and cell organization of the scaffold resembles native CNS or PNS tissue and mimics key developmental mechanisms that can be capitalized to direct cell migration and neurite outgrowth. These properties make “living scaffolds” an auspicious approach for directly replacing lost innervating fibers in native or transplanted organs and guiding the targeted integration between biofabricated organs and the native nervous system, all in service of improving the restoration of proper organ function. The tissue engineered living scaffolds play an active role in restoring the nervous system rather than being a passive substrate only. The living cells are able to modulate the biochemical milieu of the injury site to augment neuro-regeneration and serve as chaperones to guide regenerating axons. The 3D axonal tracts in the living scaffolds can also be utilized to directly replace lost neural circuitry and physically “wire in” to preserve host circuitry (FIG. 9). Biological neuromodulation is essential to mitigate cognitive, sensory, or motor deficits arising from PD, depression, drug addiction and pain disorders. Living scaffolds can also be used for biological neuro-modulation and thereby provide a smaller, permanent and self-contained alternative to conventional “hardware based” neuromodulation techniques like deep brain stimulation (DBS). Accordingly, in various embodiments the invention provides a method of treating a disease or disorder in a subject, the method comprising implanting the tissue according to claim 10 into the subject and wiring the at least one TENG or Micro-TENN to at least one native neuron of the subject.

“Stretch grown” axonal tracts referred to as tissue engineered nerve grafts (TENG) facilitate axonal regeneration across segmental nerve defects in rats (1.2 cm) as well as in ongoing porcine (5 cm) studies. TENGs of 5-10 cm length have been fabricated within 14-21 days of culture using rat embryonic dorsal root ganglia (DRG) neurons, with proof-of-concept of axonal stretch-growth in other neuronal types/sources including adult rat embryonic cortical neurons and human adult DRG neurons (cadaveric and live donors). In peripheral nerve injury models, the TENGs were found to direct and drive host axon regeneration along their length by providing topographical and biochemical cues to regenerating axons. TENGs fabricated with appropriate autonomic ganglia can be employed to project axons into target tissue cells cultured on scaffolds thereby mimicking the native architecture of axonal network that travels long distances from the autonomic ganglia to the end organ.

In a fundamentally different fabrication process, miniaturized constructs called micro-tissue engineered neural networks (micro-TENNs) are created for CNS or PNS applications. These contain millimeter to centimeter long neuronal tracts encased in a miniaturized tubular hydrogel (345-710 μm in diameter) for minimally invasive implantation (injection) into the brain or PNS. Importantly, micro-TENNs are designed to replicate the structure of axonal pathways projecting from a discrete neuronal population, thereby mimicking the gray-white matter architecture in the brain or the ganglia-axon tract architecture in the PNS/ANS. Preformed micro-TENNs fabricated to contain a population of cortical neurons with long axonal tracts could be precisely microinjected into the rat brain, where they survived, maintained their axonal architecture, and sent neurites to synaptically integrate with the host. The micro-TENN technology offers the flexibility to fabricate uni/bidirectional constructs using multiple neuronal phenotypes depending upon the nature and scale of the target neuronal circuitry. Accordingly, in various embodiments, the scaffolds of the herein described methods may comprise living scaffolds.

The preformed aligned 3D neuronal constructs or “living scaffolds” may serve as potential axonal bridges for facilitating innervation of artificial organs/tissues during fabrication as well as driving integration with the host nervous system post-implantation. The addition of correct fiber types at the proper timing ensures the desired development and maturation of the cells/tissue within bioreactors. A substrate for proper innervation post-implant would enable host mediated functional regulation of the transplanted organs based on biological feedback in a self-contained manner. For example, in MI cases, tissue engineered cardiac patches with appropriate sympathetic and parasympathetic innervation promote integration with host neurons in the area and enhance the contractility of the graft. For artificially developed whole organs like heart or pancreas, TENG-like neuronal constructs can be surgically “wired” with the host nerve supply. On the other hand, micro-TENNs can be employed for more local delivery of aligned axons to target cells especially when the innervated tissue engineered construct is being cultured in a bioreactor as described in the figures included herein. Micro-TENNs can also be used as miniaturized constructs to fabricate injectable neuromuscular junctions for preserving skeletal muscle tissue in patients with neurodegenerative diseases and long gap nerve injuries.

### Light-Activated Autonomic Ganglia as Living Scaffolds to Modulate Peripheral Organ Activity

Optogenetics involves a combination of optics and genetics that allows precise control of cellular activity by genetically modifying target cells to express light sensitive opsins which are typically ion channel, cation pumps or G-protein coupled receptors. Optogenetics has evolved as a powerful tool for light-based neuromodulation. To date, the implementation of optogenetic strategies has primarily utilizes adeno-associated virus (AAV) as vectors for vitro and in vivo studies as they provide long-term gene transfer and expression in non-proliferating tissues like nerve. AAVs are generally delivered by direct injection of AAV particles in vivo; however, in case of systemic delivery of virus, the promiscuity of AAV may lead to unwanted vector uptake by the surrounding tissue. Micro-TENN technology may be used to generate artificial biologic constructs comprised of autonomic ganglia and their projected axonal tracts to enable light-controlled modulation of end-organ function with temporal, spatial and fiber type specificity designed to replicate and expand upon the natural inputs to the system. These tissue engineered constructs act as living parallel pathways mimicking the form and function of the sympathetic and parasympathetic ganglia/fibers that naturally innervate/modulate organs (FIG. 9B). This strategy has the advantage of being highly specific, as a set of Artificial Ganglia may be created for each organ-of-interest to provide both sympathetic and parasympathetic control—in contrast to conventional organ modulation approaches based on vagus nerve stimulation that affects multiple organ systems and only modulates parasympathetic function. Accordingly, the approach disclosed herein is based on creating self-contained living axonal tracts using optogenetically-transduced autonomic ganglia in vitro followed by implantation. This provides a living parallel path near the nerve-organ interface in vivo to ensure better quality control, higher specificity of transduction and lower viral load, enhancing the overall safety. Another significant advantage is that the biologic constructs disclosed herein may modulate autonomic function without being limited by the available pool of endogenous axons, synapses, and/or neurotransmitters, which may be compromised by age, injury, or disease. Accordingly, innervated tissue generated according to the herein disclosed methods, may comprise at least one optogenetically-transducible TENG or Micro-TENN. In various embodiments, the invention further provides a method of modulating a tissue or organ of a subject, the method comprising implanting the innervated tissue comprising at least one optogenetically-transducible TENG or Micro-TENN into a subject and applying light to activate the optogenically transducible TENG or micro-TENN.

### Method of Generating Innervated Cardiac Tissue

In another aspect, the invention provides a method of generating innervated cardiac tissue, the method comprising providing a micro-column having a first end and a second end, and comprising a tubular hydrogel body and an extracellular matrix core; positioning cardiac myocyte aggregates at the first end of the micro-column and positioning sympathetic neuron aggregates at the second end of the micro-column, thereby forming a construct; and culturing the construct in vitro to promote extension of an axon of the neuron as well as the cardiac myocytes through at least a portion of the core, thereby generating innervated cardiac tissue. “Construct” here refers to the micro-column with the cell aggregates at the first and second end. Without meaning to be limited by theory, the cardiac myocyte aggregates and the sympathetic neuron aggregates grow toward the opposite end of the construct meeting in the middle and generating innervated cardiac tissue.

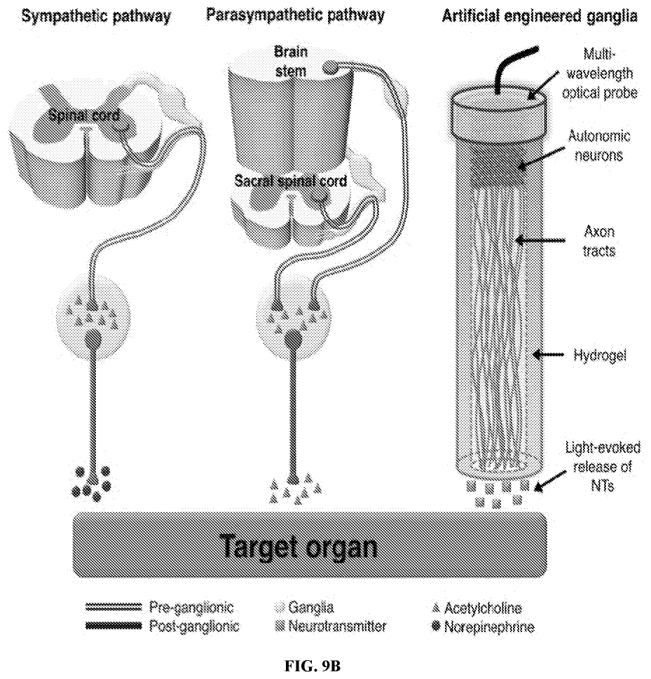

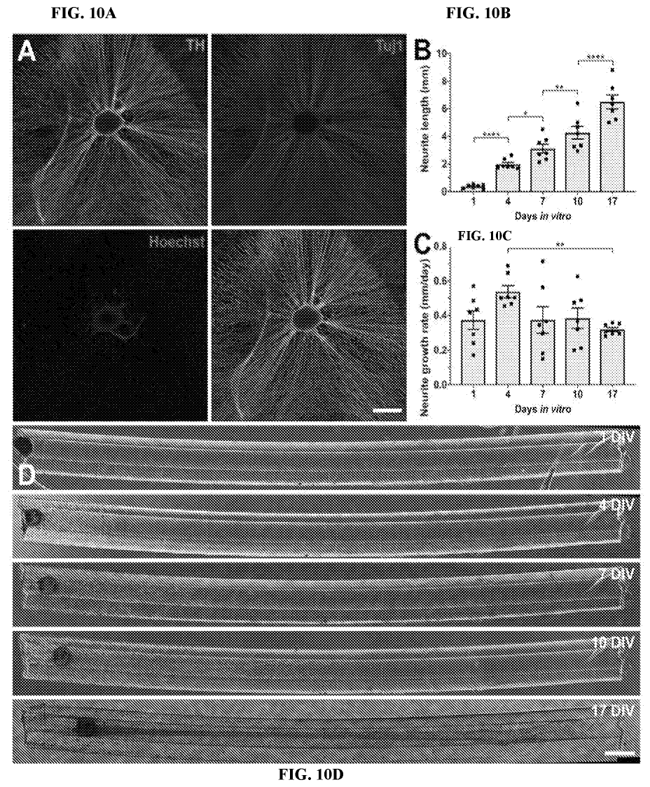

In various embodiments, generation of cardiac myocyte aggregates and sympathetic neuron aggregates is achieved by centrifugation in pyramidal wells, by way of non-limiting example, as described in Example 1. In various embodiments, positioning comprises placing the cell aggregates at the first or second end of the micro-column as shown in FIG. 10D.

In various embodiments, the tubular body comprises at least one selected from the group consisting of hyaluronic acid, chitosan, alginate, collagen, dextran, pectin, carrageenan, polylysine, gelatin and agarose. In various embodiments, the tubular body comprises methacrylated hyaluronic acid. In various embodiments, the extracellular matrix core comprises collagen, fibronectin, fibrin, hyaluronic acid, elastin, and laminin. In various embodiments, the micro-column has a length of about 3-10 mm. In various embodiments, the micro-column has an outer diameter from about 500 μm to about 1 mm. In various embodiments, the micro-column has an inner diameter from about 125 μm to about 500 μm. In various embodiments, the cardiac myocytes are mammalian cardiac myocytes. In various embodiments, the cardiac myocytes are human cardiac myocytes.

### Method of Generating Innervated Skeletal Muscle

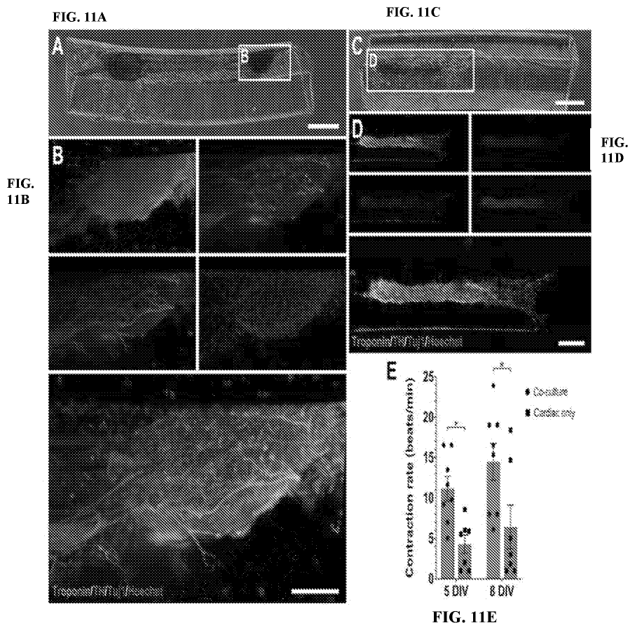

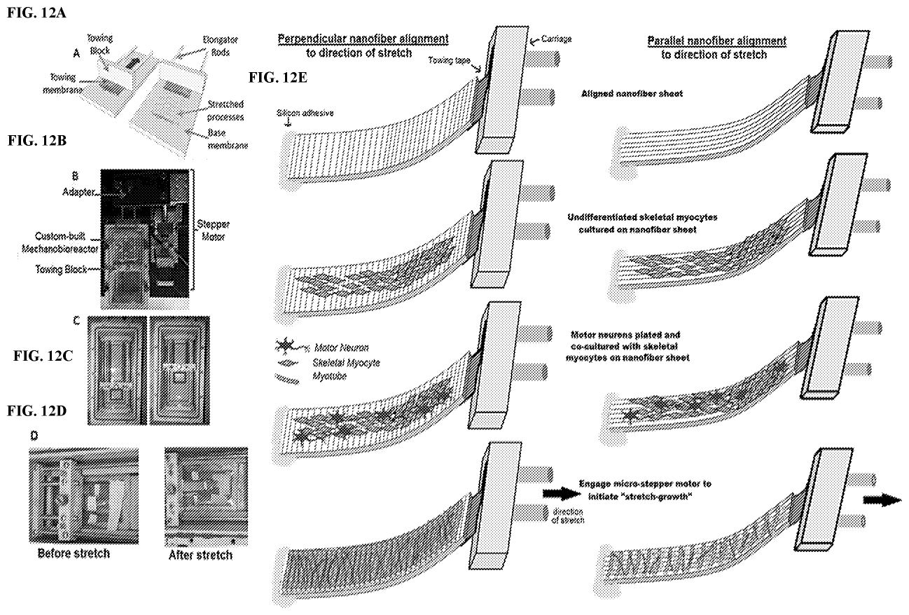

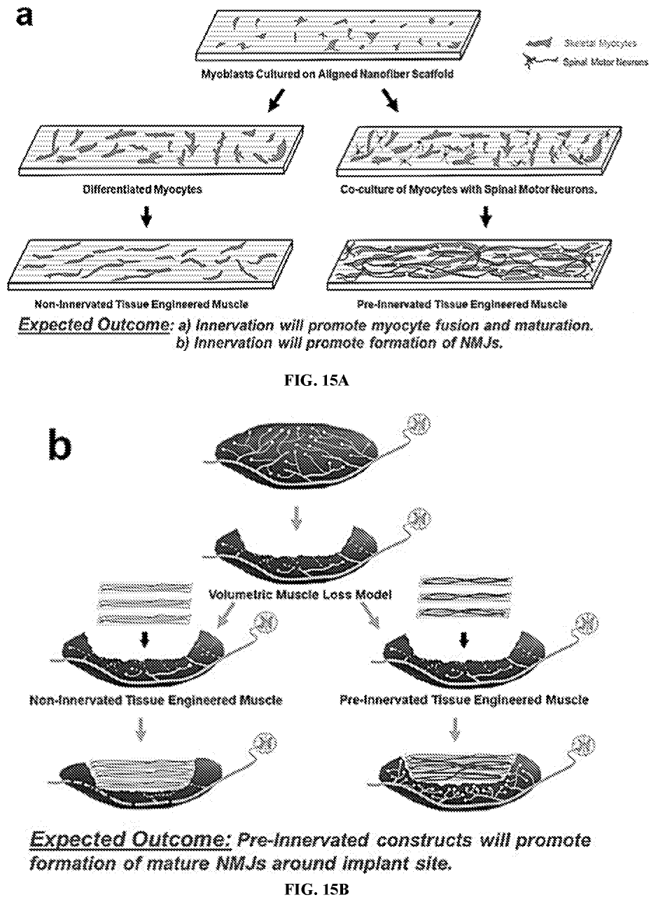

In another aspect, the invention provides a method of generating innervated skeletal muscle tissue, the method comprising culturing skeletal myocytes on a substrate comprising nanofibers aligned in a first direction, thereby forming a myocyte layer; co-culturing motor neurons on the myocyte layer; thereby generating innervated skeletal muscle tissue. The method is illustrated in Example 3 and FIGS. 15A-24F. In various embodiments, the method further comprises applying a tensile force perpendicular to the first direction. Without meaning to be limited by theory, as shown in FIGS. 12D and 12E and described in Example 3, skeletal myocytes and motor neurons grown on an aligned nanofiber substrate and stretched in a direction perpendicular to the direction of nanofiber alignment form thicker neuromuscular bundles with higher alignment than similar cells stretched in the direction of nanofiber alignment.

In various embodiments, the substrate comprises at least one selected from the group consisting of polylactic acid, poly(lactic-co-glycolic acid), polyglycolic acid and biological polymers like collagen, gelatin, hyaluronic acid and a composite of synthetic and biological polymer. In various embodiments, the substrate comprises polycaprolactone. In various embodiments, the tensile force is provided by a micro-stepper motor and is applied at a rate of about 0.1 mm/day. In various embodiments, the tensile force is applied for about 5 days to achieve a net stretch of about 0.5 mm. In various embodiments, the skeletal myocytes are mammalian skeletal myocytes. In various embodiments, the skeletal myocytes are human skeletal myocytes.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Background and Concept

The sympathetic nervous system constitutes part of the autonomic nervous system, the division involved in the functional regulation of internal organs in coordination with the central nervous system (CNS) and sensory information. In the case of the heart, sympathetic nerves regulate the circulatory and conduction systems, increasing heart rate based on the interaction between released norepinephrine and cardiac β-adrenergic receptors. The neural regulation of the heart has also been exploited for treatment of cardiac conditions through studies using vagus nerve stimulation (VNS). Sympathetic innervation has also been implicated in influencing heart size and the cell cycle stage of cardiac myocytes during development. Analogously, cardiac cells produce growth factors necessary for nerve fiber guidance and targeting. Altered or dysfunctional sympathetic innervation has been related to the pathophysiology of hypertension, arrhythmias, and heart failure.

Given the relevance of innervation for cardiac development and function, the presence of neurons must be taken into account when fabricating tissue-engineered cardiac tissues for regenerative medicine-based treatments or basic science purposes. In this study, we seek to create innervated engineered cardiac myocyte tissue in a three-dimensional (3D) environment based on the work of our research group with micro-tissue engineered neural networks (TENNs). Micro-TENNs have traditionally been comprised of CNS neuron aggregates extending aligned axonal tracts within an agarose hydrogel micro-column containing an extracellular matrix (ECM) core. In this case, we seeded micro-columns with primary sympathetic neurons derived from rat superior cervical ganglia (SCG) and primary cardiac myocytes that have been aggregated into cell clusters. Moreover, we utilized methacrylated hyaluronic acid (MeHA), a more bioactive hydrogel with readily tunable physical properties based on photocrosslinking, to create the micro-columns that encase these cells. We sought to study the innervation of 3D cardiac myocyte aggregates by sympathetic projections and the effect of their co-culture on spontaneous cardiac beating. Previous studies have shown the formation of putative synaptic connections in vitro between sympathetic neurons derived from human pluripotent stem cells and mouse neonatal ventricular myocytes. These have also demonstrated that direct physical integration was necessary for neuron-specific stimulation to have an effect on myocyte contraction. In spite of these findings, our study would represent the first instance, to our knowledge, of innervated 3D engineered cardiac tissues in vitro. Ultimately, our goal is to transduce sympathetic neurons to express light-responsive opsins to enable targeted neuronal stimulation and neuron-based modulation of cardiac contractions. These “autonomic living electrodes” may represent a new tool to interface with cardiac tissue in vivo in parallel to native nerve fibers and modulate cardiac function with temporal, spatial, and fiber type specificity. Our constructs may also serve as testbeds to understand functional relationships between neural signals and end-organ cellular activity.

### Methods

### Primary Cell Isolation and Culture:

Primary cardiac myoyctes were obtained from E16 Sprague-Dawley rat pups following previously established protocols. Briefly, hearts were dissected from pups and dissociated using 0.05% trypsin-EDTA for 10-15 min at 37° C. and manual trituration. After centrifugation, the dissociated cells were resuspended in Cardiac Media comprised of 78% DMEM-high glucose, 17% Medium-199, 4% horse serum, and 1% Pen/Strep. To form aggregates, 14 μL of a 7×10⁶ cell/mL solution were transferred to pyramidal micro-wells within a polydimethylsiloxane (PDMS) mold which were then centrifuged at 1500 rpm for 5 min, supplemented with media, and incubated overnight prior to seeding.

On the other hand, sympathetic neuron aggregates were isolated from the SCG of P0-P1 Sprague-Dawley rat pups as previously described. Briefly, postnatal pups were euthanized by hypothermia and decapitation. The SCG were located at the bifurcation of the carotid arteries at the sides of the trachea, extracted, and cleaned of pre- and post-ganglionic nerves and other debris. Pieces of the SCGs were then cultured as sympathetic neuron aggregates without any additional dissociation. SCG-only culture was done at 37° C. and 5% CO₂ using media comprised of RPMI 1640 with 0.4% Pen/Strep, 1% heat-inactivated horse serum, 10 μM of uridine/5-FDU, and 100 ng/mL nerve growth factor (NGF). When SCG were co-cultured with cardiac myocyte aggregates, the media consisted of Cardiac Media with 100 ng/mL NGF given that this growth factor is essential for sympathetic neuron survival and growth. In the case of two-dimensional (2D) planar culture, the surfaces were coated with 20 μg/mL poly-L-lysine and then 20 μg/mL laminin before seeding of cardiac myocyte and/or sympathetic aggregates.

### Three-Dimensional Culture in Hydrogel Micro-Columns:

Cardiac myocyte and sympathetic aggregates were cultured under 3D conditions using MeHA hydrogel micro-columns with lengths of 3-10 mm and outer diameter (OD) and inner diameter (ID) of 701 and 300 μm, respectively. MeHA was synthesized by the esterification of hyaluronic acid (HA) and methacrylic anhydride for ˜3.5 hr at a pH of 8.5, purified by dialysis for 5-7 days, and recovered by lyophilization. The degree of functionalization of the disaccharides in HA was evaluated as ˜44% using ¹H NMR. A MeHA solution of 3% w/v in Dulbecco's phosphate buffered saline (DPBS) with 0.05% Irgacure 2959 was drawn by capillary action into glass capillary tubes (ID: 701 μm) containing an inserted acupuncture needle (OD: 300 μm). The solution was then photocrosslinked by exposure to 10 mW/cm² ultraviolet (UV) light for 5 min to create the hydrogel. Afterwards, the needle was pulled out, and the gelled hydrogel micro-columns were removed from the capillary tube into DPBS, sterilized for 30 min with UV light, and rinsed with fresh DPBS to remove remaining free radicals. The MeHA columns were then cut to the desired length, and an ECM solution comprised of 1 mg/mL rat tail collagen type I+1 mg/mL mouse laminin in Neurobasal (pH 7.2-7.5) was added to the empty lumen and allowed to polymerize for 15 min at 37° C. Afterwards, the dishes containing micro-columns were flooded with culture media and incubated at 37° C. and 5% CO₂ until seeding.

To seed the hydrogel micro-columns, the cardiac aggregates and SCG were cut with fine forceps into pieces that could fit within the end(s) of the columns under a dissection scope. For cardiac-only cultures, one piece of a cardiac myocyte aggregate was precisely placed at one end of the micro-columns. In other cases, unidirectional sympathetic micro-columns were fabricated with only a sympathetic aggregate on one end. For cardiac-sympathetic co-cultures, the cardiac aggregate was seeded, and the other end was introduced with a sympathetic aggregate after 1 day. These micro-columns were then incubated at 37° C. and 5% CO₂ to allow for cell attachment to the ECM and/or MeHA shell and sympathetic neurite growth.

### Neurite Growth Characterization:

2D and 3D cultures were imaged with phase contrast using a Nikon Eclipse Ti-S microscope with a QiClick camera linked to Nikon Elements. Imaging was performed to quantify the length and growth rate of neurites projected from the sympathetic aggregate as a function of time. The length was determined as the distance between the longest observed neurite and the edge of the sympathetic aggregate, while the growth rate was estimated using the backward difference method. Repeated measures one-way ANOVA and Tukey's multiple comparisons tests were used to evaluate the effect of time and differences between groups, respectively, using Prism 8.1.1 (GraphPad).

### Immunocytochemistry:

At terminal time points, 2D and 3D cultures were fixed in 4% paraformaldehyde for 35 min and rinsed with 1× phosphate buffered saline (PBS). Afterwards, the cultures were permeabilized with 0.3% Triton X-100 in 4% horse serum for 60 min and then incubated overnight at 4° C. with primary antibodies in 4% horse serum. The primary antibodies used in this study were: 1) mouse anti-β-tubullin III (1/500, Sigma-Aldrich, T8578) to specifically label neurons and axons; 2) sheep anti-tyrosine hydroxylase (TH; 1/500, Abcam, ab113) to denote neurons expressing TH, the enzyme in the rate-limiting step in the biosynthesis of norepinephrine, the main neurotransmitter of sympathetic neurons; 3) rabbit anti-cardiac troponin I (1/250, Abcam, ab47003) to mark cardiac myocytes. After incubation, the cultures were exposed to secondary antibodies (Alexa-488, Alexa-568, Alexa-647; all 1/500) for 2 hr at 18-24° C., followed by 10 min of 1/10,000 Hoechst in PBS. The stained 2D and 3D cultures were imaged to assess growth, phenotype, neuronal cytoarchitecture, and innervation of cardiac myocytes using a Nikon A1RSI laser scanning confocal microscope, with their z-stacks presented here as their maximum intensity projections.

### Analysis of Spontaneous Cardiac Myocyte Contraction:

The effect of the presence of sympathetic aggregates on the rate of beating of the cardiac aggregates was analyzed by taking video recordings of co-culture (n=8) and cardiac-only (n=7) micro-columns for 1-1.5 min with the Nikon Eclipse Ti-S microscope after the cardiac aggregates had been in the micro-columns for 5 and 8 days in vitro (DIV). In this case, the sympathetic aggregates would have been inside the columns for 4 and 7 DIV, respectively, given that they were seeded one day after the cardiac aggregates. The number of beats per minute was manually quantified using Fiji. The normality of the data was confirmed using the Kolmogorov-Smirnov test, after which the effect of DIV and culture type on contraction rate was analyzed using a two-way ANOVA. Differences between groups within each time point were studied with Sidak's multiple comparisons test.

### Results

Characterization of Growth from SCG-Derived Sympathetic Neurons in Hydrogel Micro-Columns:

The results of the experiments are shown in FIGS. 10A-10D and FIGS. 11A-11E and described in the associated legends.

### Example 2

Methods

Isolation and Culture of Rat Spinal Motor Neurons

Motor neurons were harvested from the spinal cord of E16 Sprague Dawley rat embryos following previously described procedure. All harvest procedures prior to dissociation were conducted on ice. Briefly, spinal cords were extracted from the pups and digested with 2.5% 10× trypsin diluted in 1 mL L-15 for 15 mins at 37° C. The digested tissue was triturated multiple times with DNAse (1 mg/mL) and 4% BSA and centrifuged at 280 g for 10 minutes to pool all the cell suspension. Subsequently, the cell suspension was subjected to Optiprep mediated density gradient centrifugation at 520 g for 15 minutes to separate the motor neuron population. Following centrifugation, the supernatant was discarded, and cells were resuspended in motor neuron plating media consisting of glial conditioned media. Glial conditioned media was made as described earlier and supplemented with 37 ng/mL hydrocortisone, 2.2 μg/mL isobutylmethylxanthine, 10 ng/mL BDNF, 10 ng/mL CNTF, 10 ng/mL CT-1, 10 ng/mL GDNF, 2% B-27, 20 ng/mL NGF, 20 μM mitotic inhibitors, 2 mM L-glutamine, 417 ng/mL forskolin, 1 mM sodium pyruvate, 0.1 mM β-mercaptoethanol, 2.5 g/L glucose to make complete motor neuron plating media.

Mouse Skeletal Myoblast Cell Line (C2C12) Culture

C2C12 cell line was maintained in growth media comprising of DMEM-High Glucose, supplemented with 20% FBS and 1% PennStrep. The cells were allowed to reach 80% confluency before inducing differentiation through differentiation media comprising of DMEM-High Glucose supplemented with 2% NHS and 1% Penicillin-Streptomycin.

Motor Neuron-Myocyte Co-Culture on Nanofiber Sheets to Form Pre-Innervated Tissue Engineered Muscle

A 15 cm×15 cm PCL aligned nanofiber sheet was custom fabricated and purchased from Nanofiber Solutions LLC (Ohio, USA). The sheets were cut into 10 mm×5 mm pieces, placed in 24 well tissue culture plates and UV sterilized prior to coating with 20 μg/mL poly-D-lysine (PDL) in sterile cell culture water overnight. The sheets were subsequently washed thrice with PBS before coating with laminin (20 μg/mL) for 2 hours. Pre-differentiated C2C12 cells were plated on the nanofiber sheets at a concentration of 2×10⁵ cells/sheet in growth media for 24 hours before being cultured using differentiation media for 7 days in vitro (DIV) with regular changes of media. Dissociated motor neurons were plated on top of the myocyte layer at a concentration of 1×10⁵ cells/sheet and the co-culture was maintained with serum-free motor neuron media up to 14 DIV with regular changes of media. The sheets with only myocytes were also kept on serum-free motor neuron media between 7-14 DIV to maintain parity of cell culture condition between groups.

Immunofluorescence Staining of Cell Laden Nanofiber Sheets.

Samples were fixed for 35 min in 4% paraformaldehyde (EMS, Cat #15710), washed three times with 1×PBS, and permeabilized in 0.3% Triton-X100+4% Normal Horse Serum (NETS) (Sigma) for 60 min. Samples were blocked in 4% NHS (Sigma) and all subsequent steps were performed using 4% NHS for antibody dilutions. For staining of actin and AchR, samples were incubated with Alexfluor-488-conjugated phalloidin (1:200, Invitrogen, A12379) and AlexaFluor-647-conjugated bungarotoxin (1:250, Invitrogen, B35450). For assessment of motor neuron morphology and maturity, separate fixed samples were incubated with an axonal marker Tuj-1 (1:250, Abcam, ab18207) and presynaptic marker Synaptophysin (1:500, abcam, ab32127) for 16 h at 4° C. followed by Alexa Fluor-568 antibody (Life Technologies). Images were acquired using a Nikon Eclipse TI A1RSI laser scanning confocal microscope.

Quantification of Myocyte Fusion Index

Multiple replicates of MN-MYO (n=7) and MYO only (n=14) cultures were considered for measuring myocyte fusion index (MFI) as per the following equation—

$$MFI = \frac{Number of nuclei in myocytes with more than 3 nuclei}{Total number of nuclei within myocytes}$$

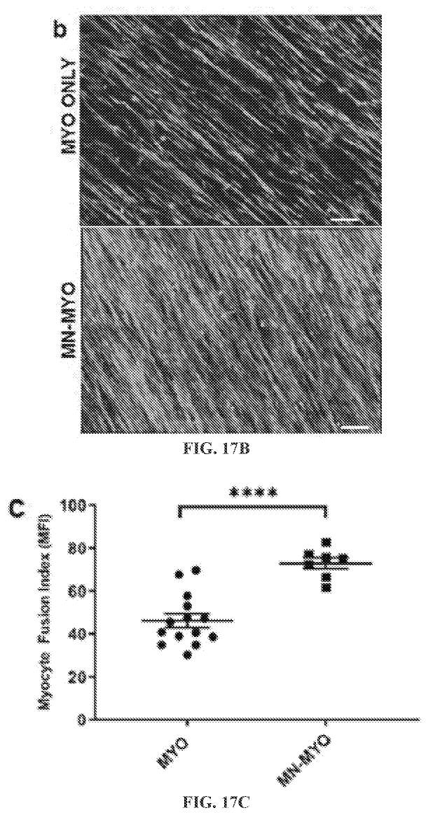

At least three 2 mm² area was considered per sample for counting MFI and the average was plotted for each sample (FIG. 17C).

Bioscaffold Implantation in Rat Model of VML

Rats had access to food and water ad libitum and were pair-housed in a colony with a 12 hr light/dark cycle.

Adult male athymic rats (RNU strain 316; Charles River Labs) weighing 280-300 g were used as subjects for this study. All procedures were carried out under aseptic conditions while the animal was under general anesthesia (1.5-2% isoflurane, 1.5 L O2) and thermal support was provided via temperature-controlled water pad. After shaving the hair of the lower left hind limb and applying a liberal coat of betadine solution, 0.25 mg of bupivacaine was administered subcutaneously along the planned incision line. Following a previously outlined procedure, a longitudinal skin incision was made along the lateral aspect of the lower leg; care was taken not to cut through the underlying fascia covering the tibialis anterior (TA) muscle. The skin was bluntly dissected from the fascia along the length of the TA. A longitudinal incision (˜1.5 cm) was made in the overlying fascia and the fascia was then gently dissected from the underlying TA muscle using a blunt probe, keeping the fascia intact for later repair. Once the muscle was exposed, a flat spatula was inserted between the tibial bone and TA muscle in order to isolate the TA/extensor digitorum longus (EDL) complex for further surgical manipulation. A mark was made 0.5 cm from the tibial tuberosity, indicating the proximal incision in the TA. A second mark was made 1.0 cm distal to the first and a 1.0 cm×0.7 cm area was outlined on the TA using a surgical caliper (Fine Science Tools, cat #18000-35). A 3 cm deep incision was made through the muscle at the proximal line and the scalpel turned parallel to the tibial bone to make a smooth cut through the muscle while following the outlined rectangle. Care was taken to avoid cutting completely through the TA or slicing the underlying EDL muscle. Once the portion of the muscle was removed, it was weighed and discarded. Deficits were repaired with 3 stacked cell laden sheets (MN-MYO, MYO), 3 stacked acellular sheets alone (SHEETS), or were not repaired (NO REPAIR). Prior to implantation, the sheets were washed thoroughly with PBS to remove any leftover media. The fascia, connective tissue, and skin were closed in layers with 8-0 prolene, 6-0 prolene, or staples, respectively. At the conclusion of the surgery, the area was cleaned with alcohol and animals were given a subcutaneous injection of sustained-release meloxicam (4 mg/kg). Animals were placed on heating pads until recovered and returned to home cages. Immunohistological Assessment of Injury Site at Acute Time Point

Freshly harvested whole anterior muscle samples (TA+EDL) were fixed in 4% paraformaldehyde (EMS, Cat #15710), submerged in 20% sucrose in 1× phosphate buffered saline (PBS, pH 7.4) for density equilibration, frozen and cryosectioned axially (20 um) across the middle portion of the graft region. Prior to staining, sections were washed three times in 1×PBS, blocked and permeablized in 4% normal horse serum (Sigma, G6767) with 0.3% Triton X-100 (Sigma, T8787) in 1×PBS for one hour. All subsequent steps were performed using blocking solution for antibody dilutions. For staining of skeletal muscle actin, samples were incubated with rabbit-anti-skeletal muscle actin (1:500, abcam, ab46805) overnight at 4° C. followed by AlexaFluor-568 antibody (1:500, Invitrogen, A10042) for two hours at room temperature. Alternatively, for staining of actin, samples were incubated with AlexaFluor-488-conjugated phalloidin (1:400, Invitrogen, A12379) for two hours at room temperature. For microvasculature staining, smooth muscle actin and endothelial cells were targeted, samples were incubated with mouse-anti-smooth muscle actin (1:500, abcam, ab7817) or rabbit-anti-CD31/PECAM1 (1:500, Novus, NB100-2284) overnight at 4° C. followed by AlexaFluor-568 antibody (1:500) or AlexaFluor-568 antibody (1:500, Invitrogen, A10087), respectively, for two hours at room temperature. For staining of satellite cells, samples were incubated with mouse-anti-Pax7 (1:10, DSHB) overnight at 4° C. followed by AlexaFluor-647 antibody (1:500, Invitrogen, A31573) for two hours at room temperature. For staining of axons, samples were incubated with sheep-anti-ChAT (1:500, abcam, ab18736) or rabbit-anti-NF200 (1:500, abcam, ab8135) overnight at 4° C. followed by AlexaFluor-568 (1:500, Invitrogen, A21099 and AlexaFluor-647 antibody (1:500, Invitrogen, A31573) respectively, for two hours at room temperature. For staining of laminin, samples were incubated with rabbit-anti-laminin (1:500, abcam, ab11575) overnight at 4° C. followed by AlexaFluor-568 antibody (1:500) for two hours at room temperature. For staining of neuromuscular junctions, samples were co-labeled with synaptophysin and bungarotoxin. Samples were incubated overnight with rabbit-anti-synaptophyhsin (1:500, abcam, ab32127) at 4° C. followed by AlexaFluor-568 antibody (1:500) and concurrently with AlexaFluor-647-conjugated bungarotoxin (1:1000, Invitrogen, B35450) for two hours at room temperature. For staining of cell nuclei, samples were incubated with Hoescht (1:10,000, Invitrogen, H3570) for 20 minutes at room temperature. Images were acquired using a Nikon Eclipse TI A1RSI laser scanning confocal microscope.

For quantitative measurement of satellite cell, micro-vessel, AchR cluster and mature NMJ density, an area of 5 mm² (5 mm long and 1 mm wide) was chosen at 100 μm from injury/implant site towards the host muscle and defined as the injury area. At least 3 cross-sections each separated by 300 μm was considered for counting and average density was plotted in the graph and compared across groups.

Statistical Analysis

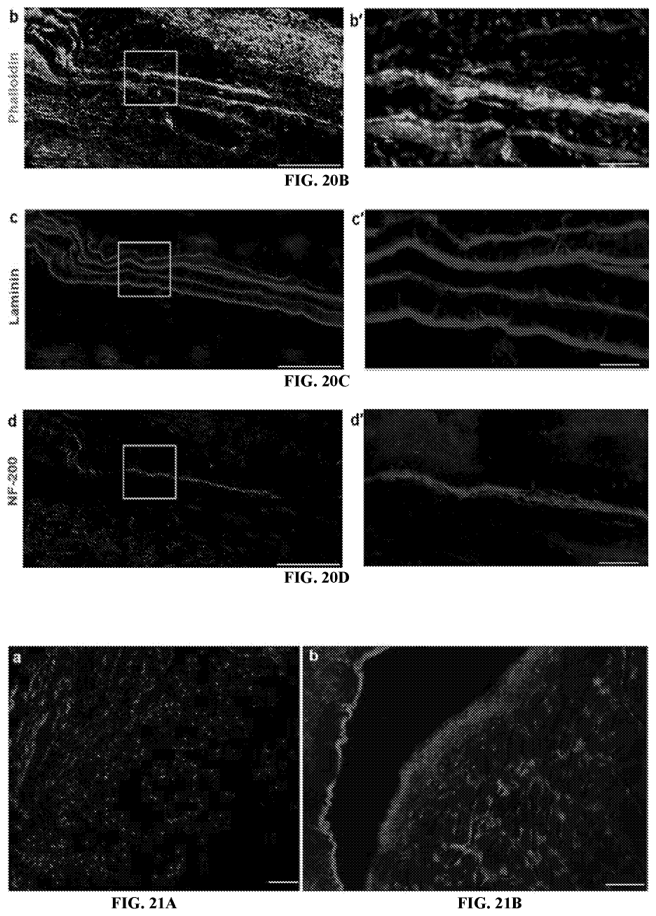

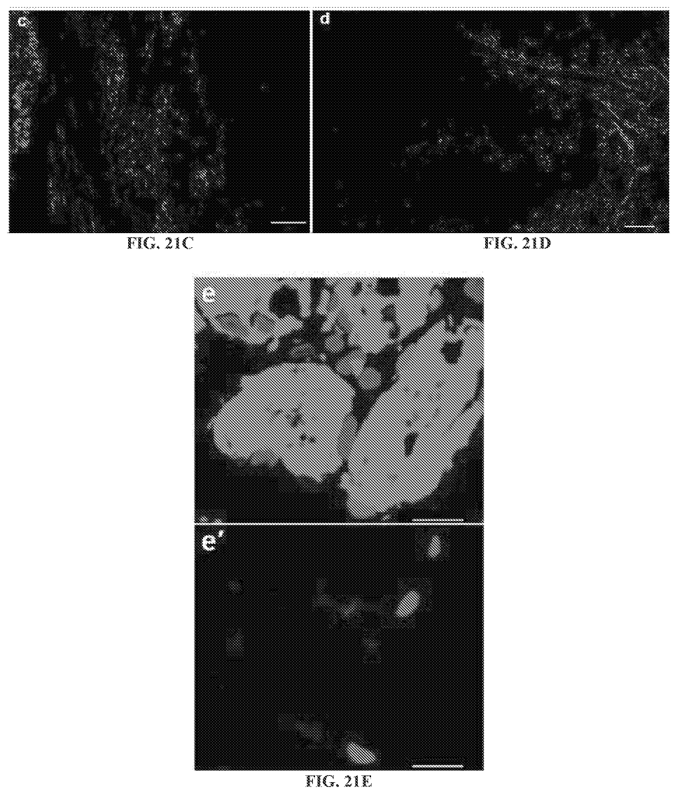

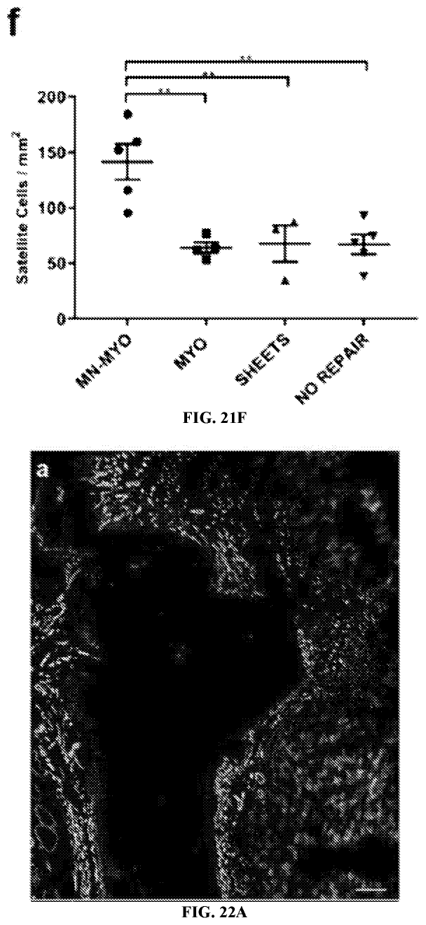

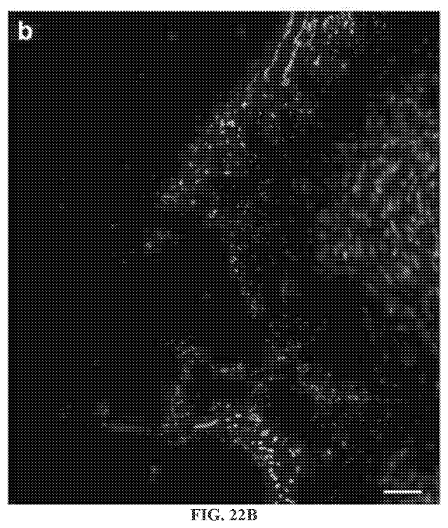

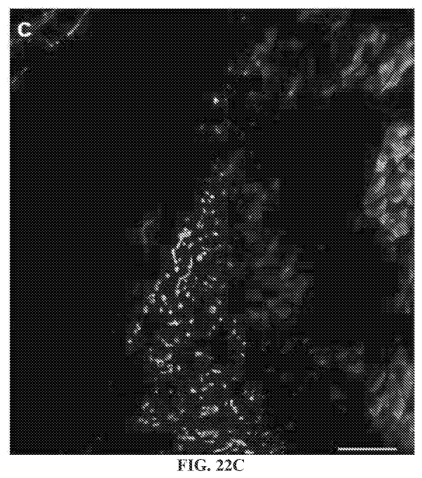

All quantifications reported in this study were performed by personnel blinded about the treatment groups. All statistical analysis was performed using GraphPad PRISM software. For comparison between two groups only (FIG. 17C), an unpaired two-tailed Student's t-test with Welch's correction was used. For comparison between multiple groups, a one-way analysis of variance (ANOVA) was performed with post hoc Tukey's adjustment with 95% Confidence Interval (FIGS. 21F, 22F, 23F, 24F). Significance was taken at p≤0.05 (*), p≤0.01 (**), p≤0.001 (***), and p≤0.0001 (****). All graphs were made in GraphPad PRISM and display mean±standard error of mean (SEM).

Results:

Pre-Innervation Promotes Myocyte Fusion and Formation of NMJs In Vitro

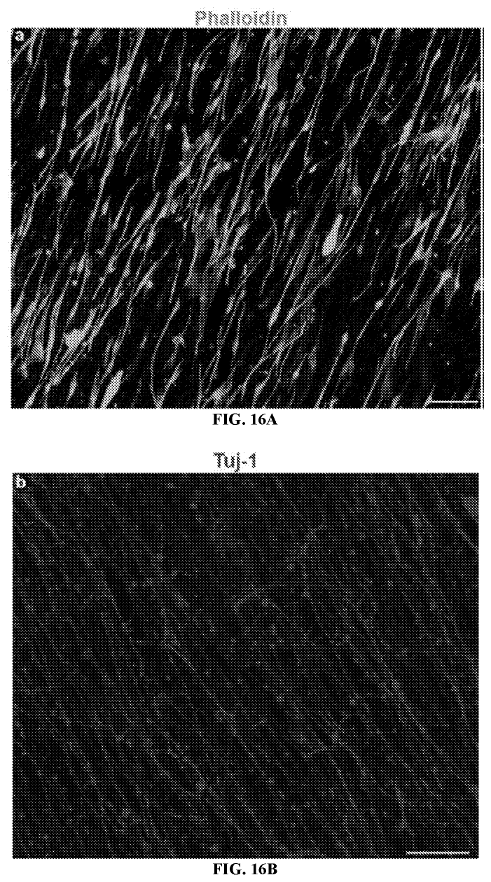

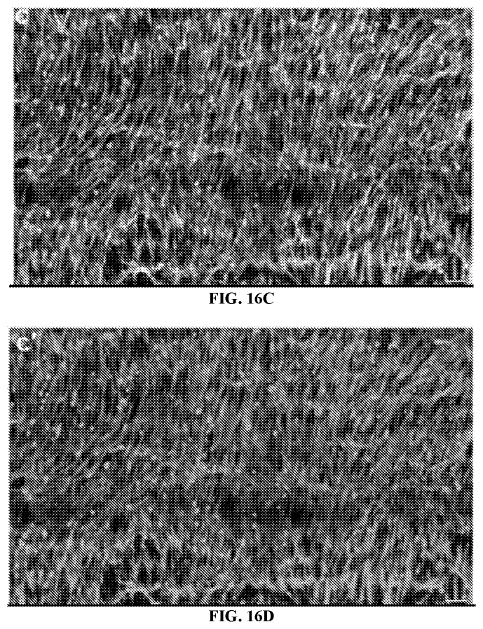

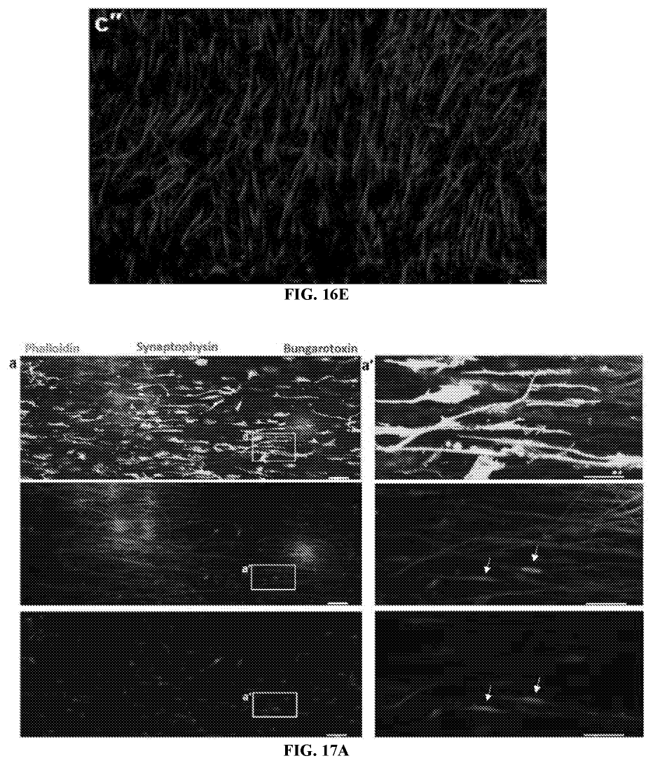

Mouse skeletal myoblast cell line C2C12 were cultured on aligned polycaprolactone (PCL) nanofiber scaffolds and allowed to differentiate. Differentiated myofibers were found to align along the direction of nanofibers as observed by staining for F-actin (FIG. 16A). Similarly, spinal motor neurons cultured on the nanofibers exhibited axons aligning along the nanofiber orientation (FIG. 16B). Subsequently, both motor neurons and myocytes were co-cultured on the nanofiber scaffolds. Motor neuron-myocyte co-culture led to formation of thick intertwined nerve-muscle bundles aligned along the nanofibers (FIGS. 16C-16D). Within 7 days of co-culture on the nanofiber scaffolds, NMJs were observed by colabelling for presynaptic marker Synaptophysin and Bungarotoxin mediated identification of post synaptic Acetylcholine Receptors (AchR) (FIG. 17A). Motor neurons were also found to promote myocyte maturation and fusion in vitro leading to significantly higher myocyte fusion index (MFI) as compared to myocyte only cultures (FIG. 17B-17C). Taken together, these data clearly demonstrates that innervation not only leads to NMJs in vitro but also facilitates myocyte maturation.

Bioscaffold Implantation in Athymic Rat Model of VML

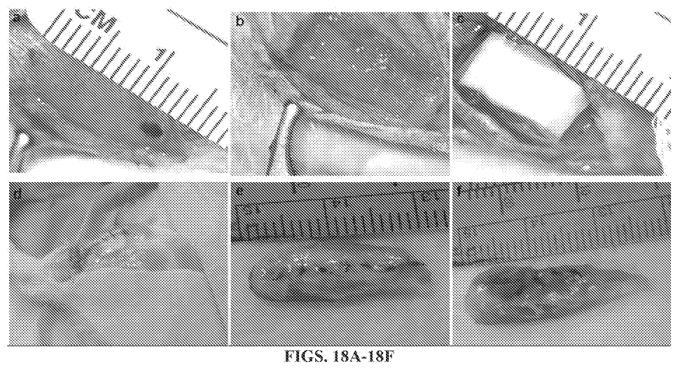

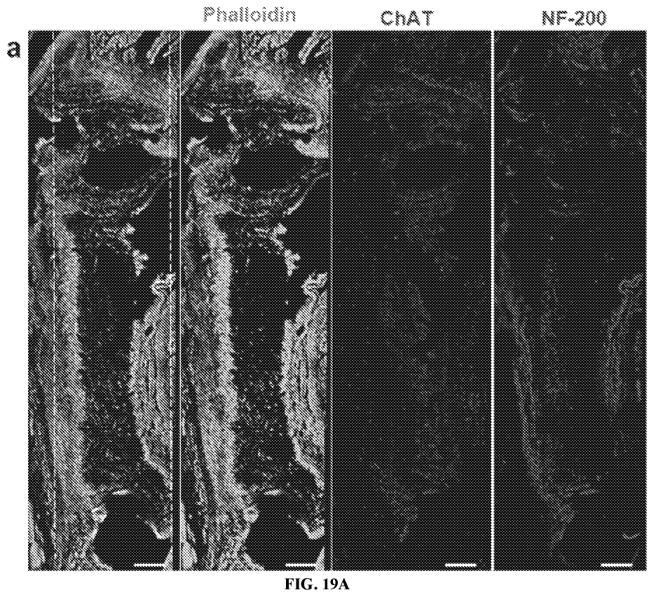

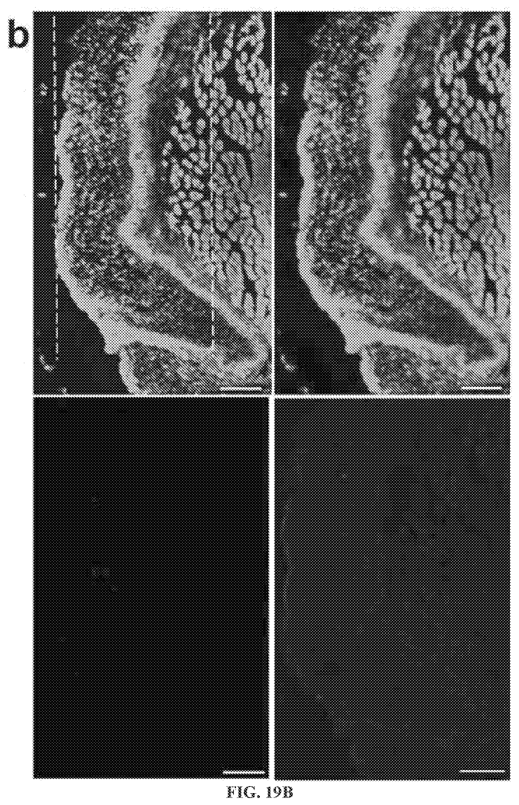

The tibialis anterior (TA) muscle of athymic rats was exposed and a 10 mm×7 mm×3 mm (length×width×depth) segment of the muscle was excised corresponding to ˜20% of gross muscle weight to create a VML model (FIGS. 18A-18B). The animals were randomized into the following repair groups: (I) three stacked nanofibrous sheets (per animal) containing co-culture of motor neurons and myocytes (MN-MYO; n=6); (II) three stacked nanofibrous sheets containing myocytes only (MYO; n=4); (III) three stacked acellular nanofibrous sheets only (SHEETS; n=3) and (IV) NO REPAIR (n=5) (FIG. 18C). At terminal time point of 7 days post implant, the nanofiber sheets were visible upon TA exposure and appeared intact (FIG. 18D). Further, the graft area in the No Repair group appeared to be recessed and atrophied as compared to the Repair groups (FIG. 18E-18F). Evaluation of acute cell survival in bioscaffolds upon implantation in VML model

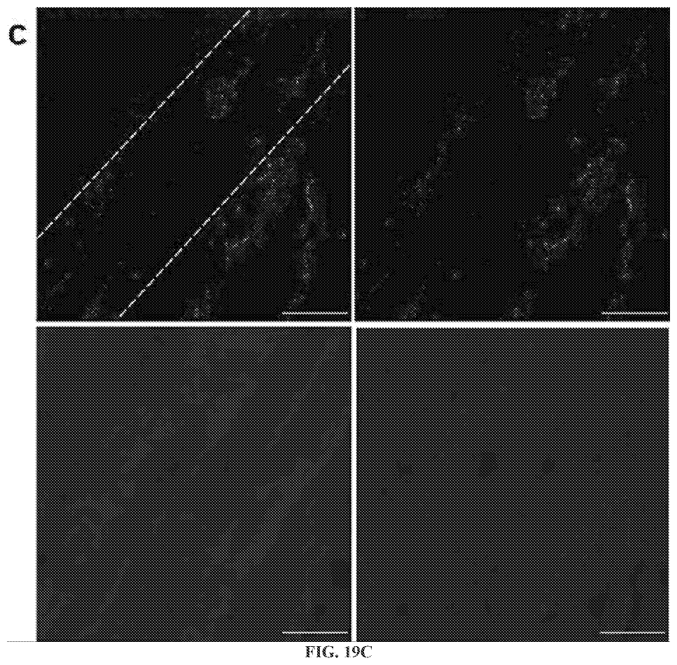

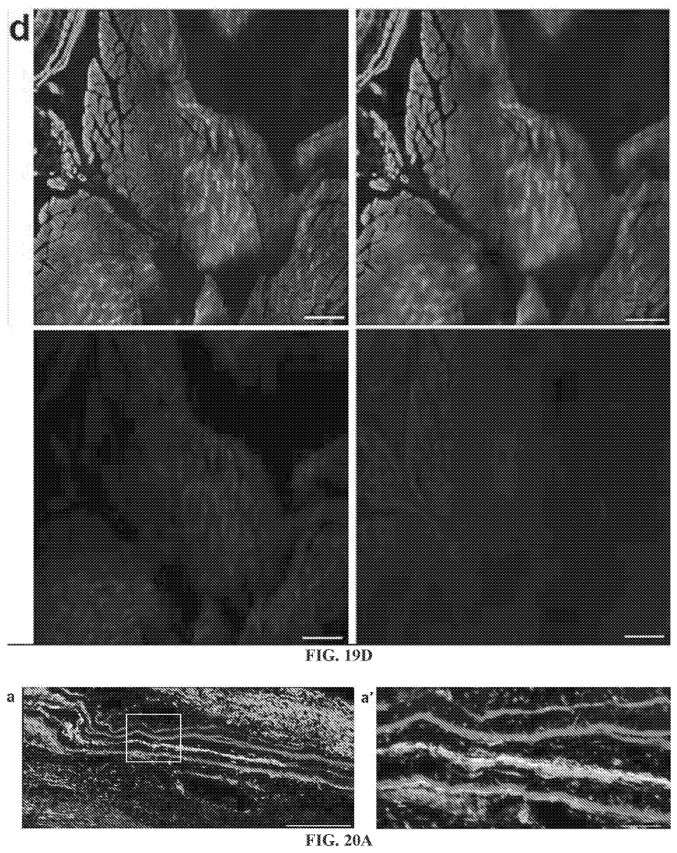

All animals were sacrificed after 7 days and the whole anterior muscle compartment of the hind limbs were fixed in paraformaldehyde. The muscles were cryopreserved, embedded in OCT, sectioned and stained. Immunohistochemical analysis of cross sections of the injury/repair sites were performed to detect implanted cells on the nanofiber sheets and assess overall muscle health. We found that the nanofiber sheets were intact after 7 days in all the animals. Myocytes positive for Phalloidin (F-actin) were observed in both MN-MYO and MYO groups (FIGS. 19A-19B). Animals implanted with SHEETS only did not show significant Phalloidin+ cells within the implant region while the NO REPAIR group was left with a gap that was eventually found to be filled with infiltrating cells (FIGS. 19C-19D). Interestingly, axons positive for motor neuron marker Choline Acetyl Transferase (ChAT) and Neurofilament (NF-200) were observed within the nanofiber sheets in animals implanted with neuron-myocyte co-cultures (MN-MYO) (FIG. 19A) whereas no axons/neurons were found within the injury/repair site in other groups. Longitudinal sections from the MN-MYO group revealed thick elongated myocytes and motor axons within the implanted nanofiber sheets (FIGS. 20A-20D). These results indicate the survival of implanted motor neurons and myocytes at acute time point following a VML repair.

Pre-Innervated Constructs Promote Satellite Cell Migration Near Injury Area

Satellite cells are resident myogenic precursor cells essential for muscle regeneration. Activation and mobilization of satellite cells to the sites of injury is a major contributor to the regenerative capability of skeletal muscle. Satellite cell migration near the injury area was observed across all groups by staining with Pax7 (FIGS. 21A-21D). Pax7⁺ nuclei located on the periphery of Skeletal Muscle Actin+ myofibers and colabelling with pan-nuclear marker DAPI were identified as satellite cells (FIG. 21E). Importantly, the pre-innervated MN-MYO group exhibited significantly higher satellite cell proliferation near the injury area as compared to other groups confirming that innervated constructs can trigger satellite cell migration and potentially facilitate muscle regeneration.

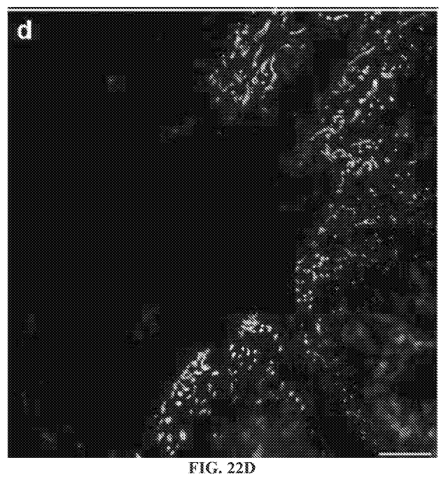

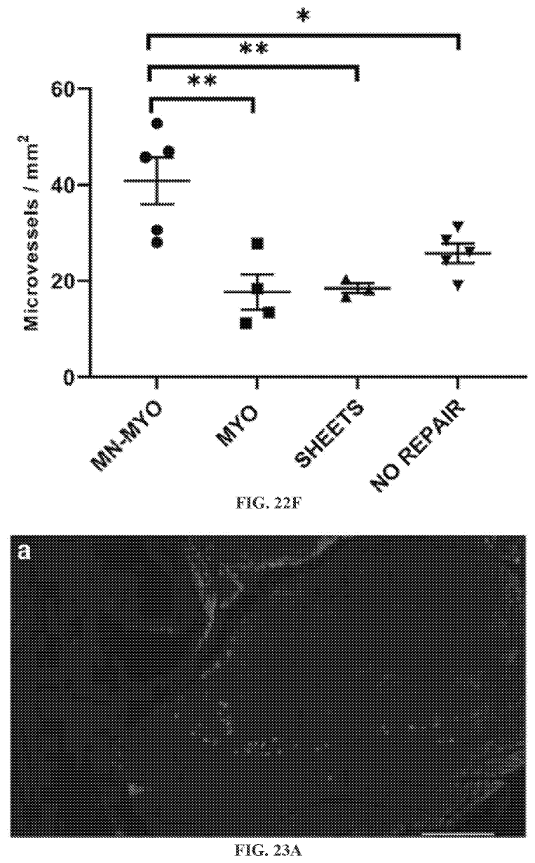

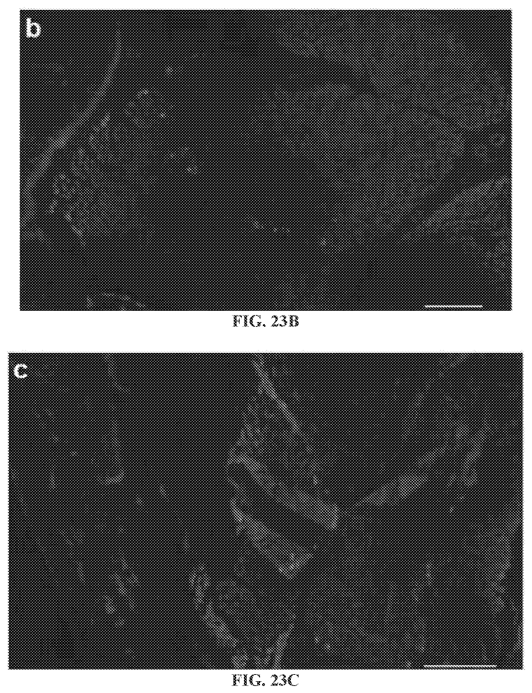

Pre-Innervated constructs lead to increased microvasculature near injury area Revascularization of the injured area/implant is critical for survival of implanted cells and integration with host vascular system. Vascularization near the injury area was evaluated by staining tissue sections with endothelial cell specific marker-CD31 and Smooth Muscle Actin (SMA) (FIGS. 22A-22F). CD31+/SMA+ structures with a visible lumen and cross-sectional area greater than 50 μm² were defined as microvessels (FIG. 22E). Although none of the groups exhibited migration of endothelial cells within the implanted sheets at this early time point (7 days), remarkably, the MN-MYO group showed presence of microvessel-like structures adjacent to the injury site while other groups had more punctate CD31+ cells (FIGS. 22A-22F). The presence of significantly higher microvasculature around the injury area in MN-MYO group suggests that innervated tissue engineered muscle constructs can potentially augment revascularization following VML repair.

Enhanced Acetylcholine Receptor (AchR) Expression Following Implantation of Pre-Innervated Constructs

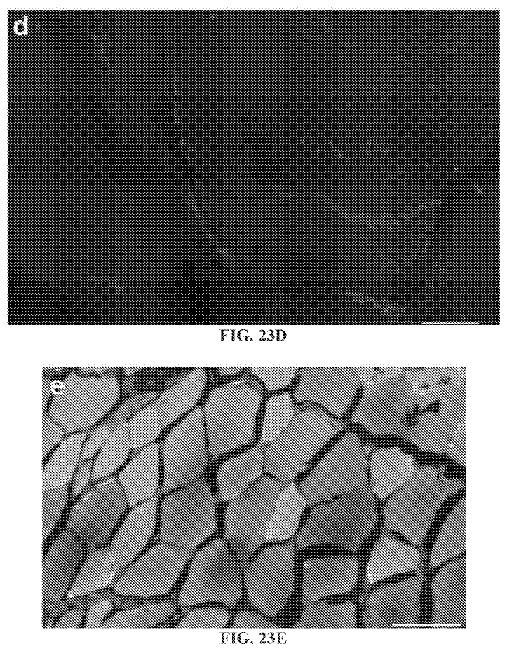

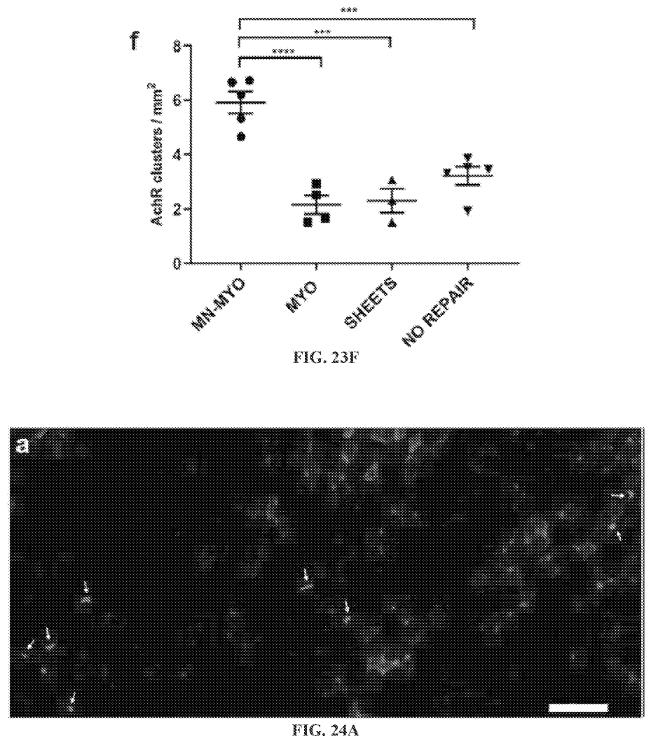

Acetylcholine Receptor (AchR) clusters have major implications in formation and maintenance of motor end plates during muscle development as well as regeneration 32,33. Indeed, bungarotoxin staining, a known marker of nAchR a7 receptors 34, showed the presence of pretzel-shaped AchR clusters around the injury area across all groups (FIGS. 23A-23E). A count of AchR cluster near the injury area revealed that the MN-MYO group had significantly more AchR clusters than the other groups (FIG. 23F).

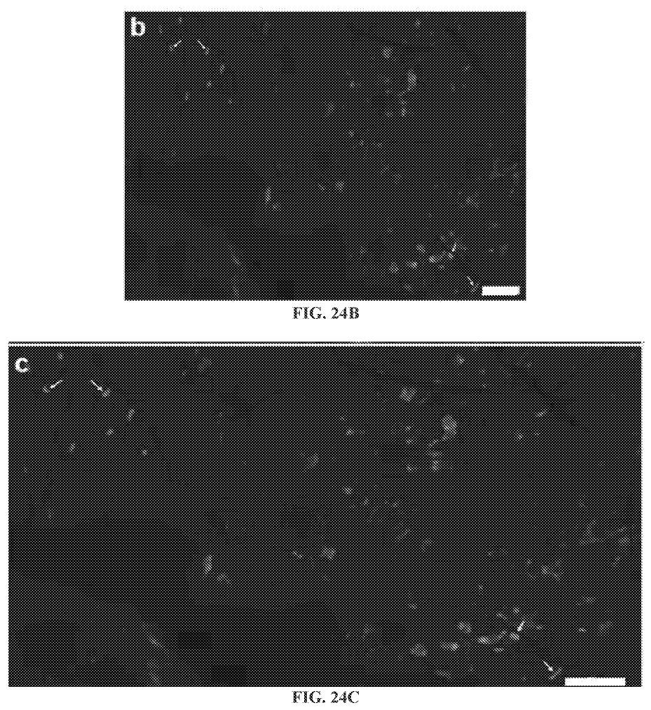

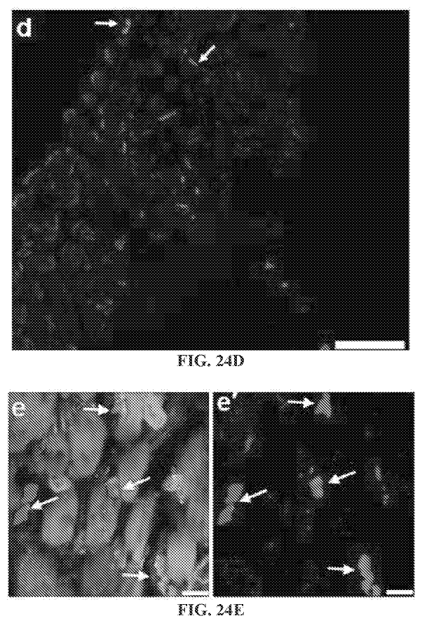

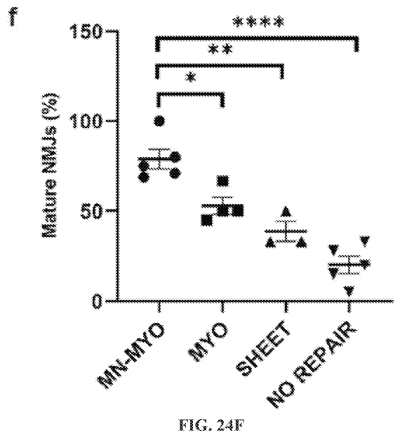

Pre-Innervated constructs promote formation of mature NMJs near injury area. Although AchR clustering is indicative of motor end plate health, they are not always the points of innervation or NMJs. Mature NMJs are indicative of muscle health and their loss has been implicated in neuromuscular degeneration associated with inflammation, denervation and atrophy 35. Mature NMJs were identified as pretzel shaped structures which were colabelled with presynaptic marker Synaptophysin and postsynaptic AchR marker (Bungarotoxin) (FIGS. 24A-24F). The percentage of AchR clusters near the injury area which were positive for Synaptophysin was calculated to quantify the amount of mature NMJs (FIG. 24F). Pre-innervated constructs (MN-MYO) were found to have significantly higher percentage of mature NMJs as compared to other groups indicating the potential role of pre-innervation in augmenting formation of mature NMJs following implantation in VML model (FIG. 24F).

Severe musculoskeletal trauma like VML is accompanied by progressive motor axotomy over several weeks, leading to denervation of the injured muscle thereby severely limiting functional recovery. Hence, appropriate somato-motor innervations remain one of the biggest challenges in fabricating a fully functional muscle. Apart from augmenting re-innervation process, tissue engineering strategies need to provide accurate cellular alignment and enable bulk muscle replacement to compensate for loss of muscle volume following VML. Aligned nanofiber scaffolds are the preferred biomaterial for muscle reconstruction since they not only promote myofibers alignment but can also be stacked to provide bulk to the engineered tissue. Aligned nanofiber scaffolds have been shown to facilitate NMJ formation in vitro as well as promote alignment of regenerating myofibers in vivo as compared to randomly oriented nanofibers. Most aligned nanofiber scaffolds used to date for VML repair are comprised of decellularized ECM or collagen which are prone to faster degradation and do not possess optimal mechanical properties to support organized myofibril regeneration. Additionally, synthetic polymer derived aligned nanofiber scaffolds are usually electropsun from polymer solutions thereby enabling scale-up and fabrication of custom designed sheets to fit the exact dimensions of an injured muscle. Although synthetic polymer derived aligned nanofibrous scaffolds have been shown to promote formation of functional NMJs in vitro, they are yet to be used as scaffolds for VML repair. The present study is the first report on using synthetic polymer based aligned nanofiber scaffolds in a rat VML model. We have used commercially available nanofiber sheets made of polycaprolactone (PCL)-which is an FDA approved slowly degrading, bioresorbable polymer. These aligned PCL nanofiber sheets were used as scaffolds for 3D motor neuron-myocyte co-culture. We studied the effect of motor neurons on myocytes in vitro and observed that motor neurons cultured on pre-differentiated skeletal myocytes led to formation of mature NMJs and promoted fusion and bundling of myocytes to form multinucleate myofibers (FIGS. 17A-17C). This is in agreement with previous report which describes that enhanced fusion and maturation of myocytes are only observed when the myocytes are allowed to fully differentiate before introduction of the motor neurons and the co-culture is maintained subsequently in serum-free conditions.

To evaluate the in vivo potential of pre-innervated constructs as a reconstructive approach to VML, we used a standardized model of VML in the rat TA muscle. Although different muscles like abdominal wall, latissimus dorsi and quadriceps femoris have been used to create a VML grade critical muscle defect, the TA muscle remains the preferred choice of researchers due to ease of surgical access and measurable functional deficit following VML. Most tissue engineering strategies towards VML repair are evaluated in small animal models. Cell based approaches generally comprise of cell lines (mouse/human) or primary cells and hence are carried out in athymic animals to allow in vivo survival and maturation of the constructs. VML has been reported to lead to over 73% motor axotomy within 7 days post injury without significant change in the number of damaged axons up to 21 days. This indicates that 7 days post injury is an appropriate acute time point to evaluate survival of implanted cells as well as study the effect of pre-innervated implants on host neuromuscular anatomy. We used T-cell deficient athymic rats to prevent immunogenic reaction to implanted mouse C2C12 cells and primary rat motor neurons. At terminal time point of 7 days post implant, the nanofiber sheets were still visible upon exposure of the TA and there were no apparent signs of immune rejection of the nanofiber sheets (FIG. 21A-21F). The aligned nanofiber sheets were highly porous (80% porosity) and our method of stacking three layers of sheets allowed exchange of nutrients and oxygen through blood perfusion thereby facilitating survival of the implanted motor neurons and myocytes. Subsequent immunohistological analysis of transverse and longitudinal sections of the muscle allowed visualization of multiple layers nanofiber sheets and confirmed the presence of long thick bundles of skeletal myocytes and motor axons on the nanofiber sheets confirming acute survival of the implanted cells (FIGS. 19A-19D and 20A-20D). In order to achieve a comprehensive understanding of the acute effects of innervation on the regenerative milieu of an injured muscle, we proceeded to investigate the density of satellite cells, microvasculature, AchR clusters and mature NMJs near the injury area.

The robust regenerative capacity of skeletal muscles can be largely attributed to the resident myogenic precursor cells called muscle satellite cells. These satellite cells lie quiescent in between the basal lamina and sarcolemma and gets activated within a few days after an injury. Activated satellite cells then differentiate to form myoblasts which fuse together to form new skeletal muscle fiber. Satellite cells can be reliably identified by paired box transcription factor Pax-7 which is expressed in both quiescent and activated stages. Although pre-vascularized tissue engineered constructs have been shown to promote satellite cell activation upon implantation in a mild muscle injury model, the effects of pre-innervation on host satellite cell population are yet to be addressed. Separate studies indicate that various neurotrophic factors like NGF and BDNF play a critical role in modulating satellite cell response within an injured muscle. For instance, exogenous treatment with BDNF was enough to recover the regenerative capacity of satellite cells in BDNF-deficient mice after skeletal muscle injury. Spinal motor neurons used in the present study for fabrication of pre-innervated constructs have been shown to secrete BDNF50. This can potentially explain the presence of significantly more Pax-7⁺ satellite cells near the injury area in MN-MYO group having pre-innervated constructs comprising of motor neurons and myocytes (FIGS. 21A-21F). However, unlike Czajka et al's (Czajka, C. A., Calder, B. W., Yost, M. J. & Drake, C. J. Implanted scaffold-free prevascularized constructs promote tissue repair. Ann. Plast. Surg. (2015)) report showing satellite cell migration within a pre-vascularized tissue engineered construct within 3 days of implantation, we did not observe any Pax-7+ satellite cells within our constructs by 7 days (FIGS. 21A-21F). This is likely due to the difference in models; VML presents a very different pathophysiology than does a mild incision injury. It is also possible that the inherent hydrophobic nature of the PCL nanofibers used here could have restricted host satellite cell infiltration. Tissue engineering strategies for VML repair demands bulk muscle reconstruction. Inadequate re-vascularization remains one of the major challenges to engineer thick skeletal muscle limiting nutrient exchange and survival of implanted cells. Pre-vascularized constructs comprising of preformed vascular networks have been shown to promote microvasculature, vascular perfusion of the graft and inosculation with host vascular system thereby significantly improving muscle regeneration following VML. Neurotrophic factors like NGF, BDNF, GDNF, NT-3 have been reported to enhance angiogenesis in different tissues like skin, heart and cartilage through receptor mediated activation or recruitment of proangiogenic precursor cells. Spinal motor neurons secrete BDNF whereas astrocytes can express a range of neurotrophic factors. It is to be noted that although we strive to obtain a pure motor neuron population, we have detected minimal glial cells in our motor neuron cultures (data not shown). We have observed that the pre-innervated constructs used in this study lead to significant increase in microvasculature near the injury area following implantation in a VML model (FIGS. 22A-22F). Although the molecular mechanisms of how pre-innervated constructs promote vascularization is the scope of future studies, it is reasonable to postulate, that neurotrophic factors from our spinal cord derived cell population (comprising of motor neurons and glia) triggered this increased microvasculature. Interestingly, despite such increased microvasculature near the injury area we did not find any evidence of endothelial cells within the implanted nanofiber sheets. Aside from the inherent hydrophobicity of PCL, the absence of endothelial cell infiltration within the graft can also indicate that the evaluation time of 7 days was too early to observe vascular integration of the construct. Muscle nicotinic AchRs are pentameric structures that are dispersed along the basal membrane of myofibers (extra-junctional) during fetal stage and progressively redistribute to form localized (junctional) pretzel shaped clusters on adult muscle. AchR clustering plays a pivotal role in skeletal muscle function and regeneration through formation of stable motor end plates thereby effecting functional restoration following severe musculoskeletal trauma. Early physical rehabilitation involving exercise has been shown to benefit patients with VML in recovering muscle force and range of motions 58. Similarly, rehabilitative exercise in conjunction with bioengineered constructs augments functional restoration in murine models of VML by promoting formation of AchR clusters and mature NMJs. One of the key findings of the present study is that pre-innervated constructs augments AchR clustering and NMJ formation. This is reflected in our results which show MN-MYO group have significantly more AchR receptor clusters and mature NMJs within 7 days of implanting in a VML model (FIGS. 23A-23F and 24A-24F). Clustering of AchRs in skeletal muscles is mainly controlled by motor innervation through secretion of neural agrin by the motor neurons. This may be a reason behind increased density of AchR clusters near the injury area following implantation of pre-innervated constructs. In a denervated muscle following injury, AchR clusters can again disperse to form extra-junctional immature receptors. This often leads to an increase in overall count of Bungarotoxin⁺ structures in an injured denervated muscle. Hence, in order to investigate if the observed increase in AchR clusters was only an injury effect, we looked for innervated AchR clusters that would indicate formation of mature NMJs and preservation of the motor end plate. The MN-MYO group was found to have significantly higher percentage of innervated AchR clusters as compared to other groups (FIGS. 24A-24F). This confirms that pre-innervated constructs promote formation of mature NMJs around the injury area at acute time points which can potentially have a significant effect in augmenting functional restoration at more chronic time points.

Although our study demonstrates the potential of pre-innervated constructs in promoting a regenerative environment following VML, several limitations exist. First, this study was conducted in athymic rats lacking an intact immune system and was terminated at an acute time point. Second, we speculate about the possible role of various neurotrophic factors in facilitating a pro-regenerative microenvironment. However, it's very likely that the physical presence of preformed motor axonal network plays a crucial role. Hence, more in depth studies are necessary to elucidate the cellular/molecular mechanisms behind the observed effects of pre-innervation in VML repair. Third, although the benefits of preformed axonal networks for in vitro tissue engineering and acute host responses are apparent in our results, longer term effects of extraneous neurons on neuromuscular integration and functional recovery are unclear. To address these shortcomings, ongoing studies in our lab are looking at effects of pre-innervated constructs on functional recovery at more chronic time points.

In summary, the present study is the first to explore the implications of pre-innervation on the regenerative microenvironment in a rat VML model at an acute time point. This is also the first report on the use of synthetic polymer derived aligned nanofiber scaffolds as an implant in a VML model. Our results indicate that pre-innervation promotes myocyte maturation in vitro, satellite cell migration and vascularization in the injury area as well as facilitates formation of mature NMJs thereby providing a favorable regenerative microenvironment for neuromuscular regeneration following VML. We believe that these findings in skeletal muscle injury model would stimulate further research into developing pre-innervated tissue engineered constructs for application in smooth muscle as well as cardiac tissue engineering. In future work, these nerve-muscle constructs may also be fabricated using cells derived from adult human stem cell sources (e.g., iPSCs), thereby making them translational as an autologous, personalized bioengineered construct. These pro-regenerative effects can potentially lead to enhanced functional neuromuscular regeneration following VML, thereby improving the levels of functional recovery following these devastating injuries.

### Example 3

### Key Findings

Motor neurons and myocytes tend to align perpendicular to the direction of stretch.
Enhanced myocyte fusion and neuromuscular bundling is observed when the cell laden scaffold is stretched perpendicular to the orientation of the nanofibers.
Mechanical “stretch” and innervation likely work synergistically to promote muscle development and maturation

### Methods

### Isolation and Culture of Rat Spinal Motor Neurons

Motor neurons were harvested from the spinal cord of E16 Sprague Dawley rat embryos following previously described procedure. All harvest procedures prior to dissociation were conducted on ice. Briefly, spinal cords were extracted from the pups and digested with 2.5% 10× trypsin diluted in 1 mL L-15 for 15 mins at 37° C. The digested tissue was triturated multiple times with DNAse (1 mg/mL) and 4% BSA and centrifuged at 280 g for 10 minutes to pool all the cell suspension. Subsequently, the cell suspension was subjected to Optiprep mediated density gradient centrifugation at 520 g for 15 minutes to separate the motor neuron population. Following centrifugation, the supernatant was discarded, and cells were resuspended in motor neuron plating media consisting of glial conditioned media. Glial conditioned media was made as described earlier and supplemented with 37 ng/mL hydrocortisone, 2.2 μg/mL isobutylmethylxanthine, 10 ng/mL BDNF, 10 ng/mL CNTF, 10 ng/mL CT-1, 10 ng/mL GDNF, 2% B-27, 20 ng/mL NGF, 20 μM mitotic inhibitors, 2 mM L-glutamine, 417 ng/mL forskolin, 1 mM sodium pyruvate, 0.1 mM β-mercaptoethanol, 2.5 g/L glucose to make complete motor neuron plating media.

### Mouse Skeletal Myoblast Cell Line (C2C12) Culture

C2C12 cell line was maintained in growth media comprising of DMEM-High Glucose, supplemented with 20% FBS and 1% PennStrep. The cells were allowed to reach 80% confluency before inducing differentiation through differentiation media comprising of DMEM-High Glucose supplemented with 2% NHS and 1% Penicillin-Streptomycin.

### Stretch Growth of Motor Neuron-Myocyte Co-Culture on Nanofiber Sheets

A 15 cm×15 cm PCL aligned nanofiber sheet was cut into 25 mm×5 mm pieces and placed within our custom designed mechanical bioreactors as described in FIG. 12D. The entire setup was UV sterilized prior to coating with 20 μg/mL poly-D-lysine (PDL) in sterile cell culture water overnight. The sheets were subsequently washed thrice with PBS before coating with laminin (20 μg/mL) for 2 hours. Pre-differentiated skeletal myocytes were plated on the nanofiber sheets at a concentration of 2×10⁵ cells/sheet in growth media for 24 hours before being cultured using differentiation media for 7 days in vitro (DIV) with regular changes of media. On Day 7, dissociated motor neurons were plated on top of the myocyte layer at a concentration of 1×10⁵ cells/sheet and the co-culture was maintained with serum-free motor neuron media up to Day 9 with regular changes of media. On Day 9, the cell laden nanofiber sheets were stretched as illustrated in FIG. 12E at a rate of 0.1 mm/day for 5 days to achieve a net stretch of 0.5 mm.

### Results

### Mechanical Properties of Nanofiber Scaffolds According to Direction of Stretch

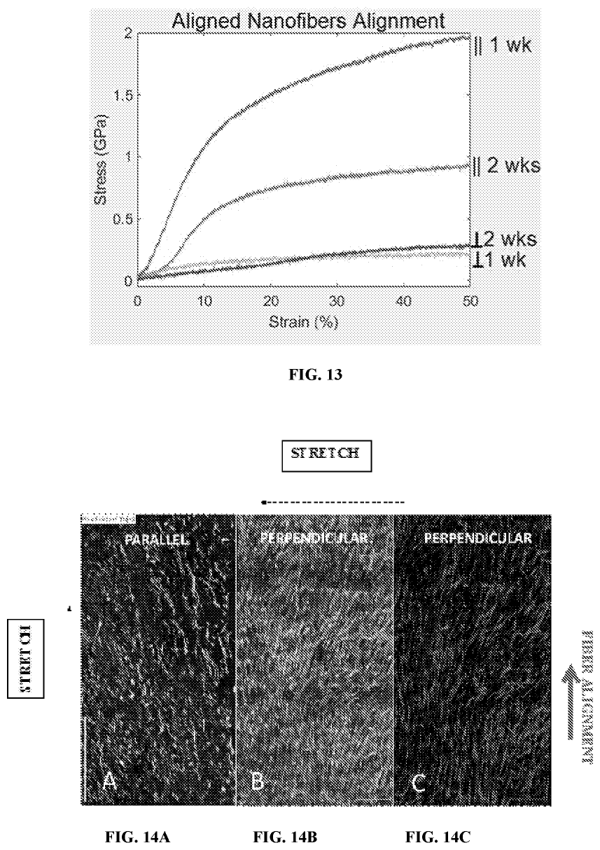

The nanofiber sheets exhibited higher Young's Modulus when stretched parallel to the fiber alignment as compared to when stretched perpendicular to fiber alignment. There was almost 50% reduction in Youngs's Modulus of the nanofiber sheets after being soaked in water for 2 weeks when stretched parallel to fiber alignment. Mechanical properties in response to tensile forces perpendicular to fiber alignment did not significantly change over 2 weeks of being soaked in water (FIG. 13).

### Cellular Response to Mechanical Stretch

Motor neurons and myocytes co-cultured on nanofiber sheets were observed to orient along the direction of fiber alignment before initiation of stretch. When stretched in a direction perpendicular to fiber alignment, the motor axons and myocytes formed thick neuromuscular bundles along the nanofibers. However, tensile force along the direction of fiber alignment resulted in disperse and less aligned population of motor neuron and myocytes.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of generating innervated cardiac tissue, the method comprising:
a) isolating cardiac myocytes;
b) culturing the cardiac myocytes on a first scaffold;
c) isolating and culturing sympathetic ganglia and parasympathetic neurons from cervical ganglia and intracardiac ganglia;
d) co-culturing parasympathetic neurons with the cardiac myocytes on the first scaffold;
e) culturing the sympathetic ganglia on a second scaffold adjacent to the first scaffold, thereby forming a construct;
f) maturing the construct in a bioreactor; thereby generating innervated cardiac tissue.

2. A method of generating innervated tissue engineered pancreatic tissue, the method comprising:
a) isolating pancreatic acinar and beta islet cells;
b) culturing the pancreatic acinar cells and beta islet cells on a first scaffold;
c) isolating and culturing sympathetic ganglia and parasympathetic neurons;
d) co-culturing parasympathetic neurons with the pancreatic acinar cells and beta islet cells on the first scaffold;
e) culturing the sympathetic ganglia on a second scaffold adjacent to the first scaffold, thereby forming a construct;
f) maturing the construct in a bioreactor;
thereby generating innervated pancreatic tissue.

3. A method of generating innervated intestinal tissue, the method comprising:
a) isolating intestinal smooth muscle cells;
b) culturing the intestinal smooth muscle cells on a first scaffold;
c) isolating and culturing enteric neurons;
d) co-culturing enteric neurons with the intestinal smooth muscle cells on the first scaffold, thereby forming a construct;
e) maturing the construct in a bioreactor; thereby generating innervated intestinal tissue.

4. A method of generating innervated salivary gland tissue, the method comprising:
a) isolating salivary acinar cells;
b) culturing the salivary acinar cells on a first scaffold;
c) isolating and culturing sympathetic and parasympathetic neurons;
d) culturing sympathetic neurons on a second scaffold, culturing parasympathetic neurons on a third scaffold, wherein the second scaffold and the third scaffold are adjacent to the first scaffold, thereby forming a construct;
e) maturing the construct in a bioreactor; thereby generating innervated salivary gland tissue.

5. A method of generating innervated skeletal muscle tissue, the method comprising:
a) isolating skeletal myocytes;
b) culturing the skeletal myocytes on a first scaffold to form myofibers;
c) isolating spinal motor neurons;
d) co-culturing the motor neurons with the myofibers on the first scaffold, thereby forming a construct;
e) maturing the construct in a bioreactor; thereby generating innervated skeletal muscle tissue.

6. A method of generating innervated spleen tissue, the method comprising:
a) isolating sympathetic neurons;
b) culturing the sympathetic neurons on a first scaffold while allowing axonal growth to an adjacent second scaffold;
c) isolating splenocytes;
d) co-culturing the splenocytes on the first scaffold with the sympathetic neurons;
e) maturing the construct in a bioreactor; thereby generating innervated spleen tissue.

7. A method of generating innervated bladder tissue, the method comprising:
a) isolating bladder smooth muscle cells and urothelial cells;
b) co-culturing the bladder smooth muscle cells and the urothelial cells on a first scaffold;
c) isolating sympathetic neurons and parasympathetic neurons;
d) culturing the sympathetic neurons on a second scaffold and the parasympathetic neurons on a third scaffold, wherein the second and third scaffolds are adjacent to the first scaffold, thereby forming a construct;
e) maturing the construct in a bioreactor; thereby generating innervated bladder tissue.

8. The method according to claim 1, wherein at least one scaffold comprises a living scaffold.

9. Innervated tissue generated according to claim 1.

10. The innervated tissue according to claim 9, comprising at least one TENG or Micro-TENN.

11. A method of treating a disease or disorder in a subject, the method comprising implanting the tissue according to claim 9 into the subject.

12. A method of treating a disease or disorder in a subject, the method comprising implanting the tissue according to claim 10 into the subject and wiring the at least one TENG or Micro-TENN to at least one native neuron of the subject.

13. The innervated tissue according to claim 10, wherein the at least one TENG or Micro-TENN is an optogenetically-transducible TENG or Micro-TENN.

14. A method of modulating a tissue or organ of a subject, the method comprising implanting the innervated tissue of claim 13, into the subject and applying light to activate the optogenically transducible TENG or micro-TENN.

15. A method of generating innervated cardiac tissue, the method comprising:
a) providing a micro-column having a first end and a second end, and comprising a tubular hydrogel body and an extracellular matrix core;
b) positioning cardiac myocyte aggregates at the first end of the micro-column and positioning sympathetic neuron aggregates at the second end of the micro-column, thereby forming a construct;
c) culturing the construct in vitro to promote extension of an axon of the neuron as well as the cardiac myocytes through at least a portion of the core, thereby generating innervated cardiac tissue.

16. The method according to claim 15, wherein the tubular body comprises at least one selected from the group consisting of hyaluronic acid, chitosan, alginate, collagen, dextran, pectin, carrageenan, polylysine, gelatin and agarose.

17. The method according to claim 16, wherein the tubular body comprises methacrylated hyaluronic acid.

18. The method according to claim 15, wherein the extracellular matrix core comprises collagen, fibronectin, fibrin, hyaluronic acid, elastin, and laminin.

19. The method according to claim 15, wherein the micro-column has a length of about 3-10 mm.

20. The method of claim 15, wherein the micro-column has an outer diameter from about 500 μm to about 1 mm.

21. The method of claim 15, wherein the micro-column has an inner diameter from about 125 μm to about 500 μm.

22. A method of generating innervated skeletal muscle tissue, the method comprising:
a) culturing skeletal myocytes on a substrate comprising nanofibers aligned in a first direction, thereby forming a myocyte layer;
b) co-culturing motor neurons on the myocyte layer; thereby generating innervated skeletal muscle tissue.

23. The method according to claim 22, wherein the substrate comprises polycaprolactone.

24. The method according to claim 22, further comprising:
a) applying a tensile force perpendicular to the first direction.

25. The method according to claim 24, wherein the tensile force is applied at a rate of about 0.1 mm/day.

26. The method according to claim 25, wherein the tensile force is applied for about 5 days to achieve a net stretch of about 0.5 mm.

27. The method according to claim 15, wherein the cardiac myocytes are mammalian cardiac myocytes.

28. The method according to claim 15, wherein the cardiac myocytes are human cardiac myocytes.

29. The method according to claim 22, wherein the skeletal myocytes are mammalian skeletal myocytes.

30. The method according to claim 22, wherein the skeletal myocytes are human skeletal myocytes.

31. A method of treating a muscle injury in a subject in need thereof, the method comprising contacting the muscle injury with innervated skeletal muscle tissue generated by the method according to claim 22.

32. A method of modeling development, maturation, function, injury, and/or disease, the method comprising using the innervated engineered tissue generated according to claim 1 as an in vitro testbed.
